(19) United States
(12) Patent Application Publication
(10) Pub. No.: US 2022/0079853 A1
(43) Pub. Date: Mar. 17, 2022

(54) SKIN CARE COMPOSITIONS AND METHODS

(71) Applicant: Alessandro VAROTTO, Santa Monica, CA (US)

(72) Inventor: Alessandro VAROTTO, Santa Monica, CA (US)

(21) Appl. No.: 17/313,613

(22) Filed: May 6, 2021

Related U.S. Application Data
(63) Continuation of application No. 16/638,063, filed on Feb. 10, 2020, now abandoned.
(60) Provisional application No. 62/544,064, filed on Aug. 11, 2017.
(60) Provisional application No. 62/565,834, filed on Sep. 29, 2017.
(60) Provisional application No. 62/580,309, filed on Nov. 1, 2017.

(51) Int. Cl. A61K 8/36; A61Q 19/00; A61Q 19/06; A61Q 19/08

(52) U.S. Cl. CPC A61K 8/361; A61Q 19/007; A61K 2800/88; A61Q 19/08; A61Q 19/06

(57) ABSTRACT

Skin care compositions, methods, and kits are provided. In some embodiments, the skin care compositions include one or more oils that include ximenynic acid, and one or more oils that include linolenic acid, and/or one or more oils that include linoleic acid. In certain embodiments, methods of using the aforementioned skin care formulations are provided for the treatment of acne, psoriasis, eczema, or dermatitis.

### CROSS-REFERENCE TO RELATED APPLICATION

This application claims the benefit of U.S. Provisional Patent Application No. 62/544,064 filed on Aug. 11, 2017, and U.S. Provisional Patent Application No. 62/565,834 filed on Sep. 29, 2017, and U.S. Provisional Patent Application No. 62/580,309 filed on Nov. 1, 2017. All of these applications are incorporated herein by reference.

### FIELD OF THE INVENTION

The instant disclosure relates to compositions, methods, and kits for skin care.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

There are numerous skin care products available on the market. Many of these products are not “natural,” as they include ingredients that have been chemically processed and/or synthesized. Additionally, most skin care formulations that are meant to moisturize take a relatively long time to absorb into skin, and therefore leave a residue that can be unsightly, sticky or greasy, and is harmful to clothing. Lotions, oils, and other products that moisturize while simultaneously providing sun protection are especially likely to have these characteristics.

There is clearly a need in the art for skin care compositions/formulations that improve the condition of skin while not leaving a greasy or sticky residue. Formulations that can achieve these results while also providing sun protection and/or antimicrobial and/or antiaging properties are especially needed.

### SUMMARY OF THE INVENTION

Various embodiments provide a composition for topical application to a subject's skin. In some embodiments the composition includes one or more oils that include ximenynic acid, and one or more oils that include linolenic acid, and/or one or more oils that include linoleic acid. In some aspects, linolenic acid is alpha linolenic acid; in some aspects, linolenic acid is gamma linolenic acid; and in some aspect linolenic acid is either alpha or gamma linolenic acid, or a mixture of both. In certain embodiments, the composition includes an oil that includes ximenynic acid (or ximenynic acid is extracted from), an oil that includes linolenic acid (or linolenic acid is extracted from), and an oil that includes linoleic acid (or linoleic acid is extracted from). In certain embodiments, the oil that includes ximenynic acid is chosen from blunt sandalwood fruit kernel, Australian sandalwood nut oil, and native cherry seed oil. In some embodiments, the oil that includes ximenynic acid is sandalwood nut oil. In certain embodiments, the oil that includes ximenynic acid is native cherry seed oil. In some embodiments, the oil that includes linolenic acid is chosen from sea buckthorn seed oil, black currant seed oil, and borage seed oil. In some embodiments, the oil that includes linolenic acid is sea buckthorn seed oil. In certain embodiments, the oil that includes linolenic acid is borage seed oil. In certain embodiments, the composition includes an oil that includes linoleic acid. In some embodiments, the oil that includes linoleic acid is chosen from salicornia oil, safflower oil, and red raspberry seed oil. In certain embodiments, the oil that includes linoleic acid is red raspberry seed oil. In some embodiments, the oil that includes linoleic acid is salicornia oil.

One embodiment provides a composition including, but are not limited to, sandalwood nut oil and sea buckthorn oil. Another embodiment provides a composition consisting essentially of sandalwood nut oil and sea buckthorn oil. Another embodiment provides a composition including, but are not limited to, sandal wood nut oil, sea buckthorn seed oil, and red raspberry seed oil. Yet another embodiment provides a composition consisting essentially of sea buckthorn seed oil, red raspberry seed oil, and sandal wood nut oil, and exemplary relative weight percentages of the three oils, respectively, are 45-65% for sea buckthorn seed oil, 23-43% for red raspberry seed oil, and 2-22% for sandalwood nut oils, w/w, (with a totality of three oils about 100%). One other embodiment provides a composition including, but are not limited to, sea buckthorn oil, borage seed oil and sandalwood nut oil.

Some embodiments provide a composition including two, three, four, five, six or more of sea buckthorn seed oil, sandalwood seed oil (also known as Sandalwood nut oil), tea tree oil, frankincense oil, geranium oil, grapefruit oil, and lavender oil. In some aspects, the composition includes sea buckthorn seed oil, sandalwood seed oil (also known as Sandalwood nut oil), tea tree oil, frankincense oil, geranium oil, grapefruit oil, and lavender oil.

Yet other embodiments provide a skin scrub composition including, but are not limited to, ground coffee, sea salt, and sugar. Other embodiments provide a skin scrub composition consisting essentially of ground coffee, sea salt, and sugar.

Various embodiments provide applying any of the formulations described above to a subject's skin. In some embodiments, the region of the subject's skin to which the composition is applied includes one or more characteristics such as a lesion of acne, dryness, a lesion of psoriasis, a lesion of eczema, a lesion of dermatitis, a wrinkle, a lesion of cellulitis, or a combination thereof In certain embodiments, the subject is a human. In some embodiments, one of the characteristics of the subject's skin is a lesion of acne. In some embodiments, one of the characteristics of the subject's skin is a lesion of psoriasis.

Various embodiments provide a kit for treating one or more skin condition. In some embodiments, the kit includes a composition described herein above and instructions for using the composition to treat the skin condition. In certain embodiments, the skin condition is dryness, psoriasis, eczema, dermatitis, a wrinkle, or cellulitis. In certain embodiments, the kit further includes an applicator such as a dropper or an absorbent material.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 illustrates, in accordance with various embodiments of the invention, a 6-day time lapse comparison between a slice of an apple that was untreated (left side of each photograph) and a slice of the same apple that was treated once on day 1 with an oil-based skin care composition containing sandalwood nut oil, sea buckthorn seed oil, and red raspberry seed oil (right side of each photograph). The progression over time is from left to right. It is evident that the untreated slice of apple oxidized and degraded much more quickly than the slice of apple treated with the protective skin care composition.

FIG. 2 illustrates, in accordance with various embodiments of the invention, a closer view of the two apple slices from day 6 of the experiment described in FIG. 1. The oxidation and microbial degradation of the untreated apple slice on the left can be clearly seen. The apple slice on the right has been largely protected from oxidation and microbial degradation, demonstrating the strong antioxidant and antimicrobial properties of the composition.

FIG. 3 illustrates, in accordance with various embodiments of the invention, a comparison between one hand (left) that was treated with an oil-based skin care composition containing sandalwood nut oil, sea buckthorn seed oil, and red raspberry seed oil, and another hand (right) that was treated with the same amount of coconut oil. After 20 seconds, each hand was covered evenly with the same amount of flour. Subsequently, hands were shaken, inverted, and lightly rubbed to remove excess. It is clear that the hand on the left had significantly less flour remaining, indicating that the skin care composition applied to the hand on the left absorbed into the hand much more quickly and left less sticky residue than the composition applied to the right hand. The same experiment was repeated using jojoba oil, argan oil, and confectioner sugar as controls, affording similar results. Using each of these experiments, the hand with the oil-based composition containing sandalwood nut oil, sea buckthorn seed oil, and red raspberry seed oil resulted in less flour sticking to the hand than with jojoba oil, argan oil, and confectioner sugar.

FIG. 4 illustrates, in accordance with various embodiments of the invention, a comparison between a slice of cantaloupe treated with a with an oil-based skin care composition containing, sandalwood nut oil, sea buckthorn seed oil, and red raspberry seed oil and one untreated slice. It is evident that the treated slice displays insect repellant properties. For the treated slice, a few drops of the skin care composition was applied on the surface, then it was not touched again. During observation of the slices over the course of 5 days, a group of 5 fruit flies landed periodically on the slice that was not treated. However, flies never landed on the slice that was treated.

FIG. 5A-5I illustrate, in accordance with various embodiments of the invention, results of bacterial growth experiments on agar plates, demonstrating the antimicrobial effect of ingredients included in certain formulations described herein. In each of the agar plates shown, the following procedure was performed. An index finger was thoroughly washed with water and dried. The finger was rubbed vigorously back and forth 3 times on the surface of a dirty kitchen floor that had not been cleaned for 24 hours. For each experiment the index finger was subsequently treated with ingredients as follows: 5A) sea buckthorn seed oil; 5B) sandalwood seed oil (also known as Sandalwood nut oil); 5C) tea tree oil; 5D) frankincense oil; 5E) geranium oil; 5F) grapefruit oil; and 5G) lavender oil. In a control experiment, represented in 5H) the contaminated finger wasn't treated with any product. In the last experiment, represented in 5I), the finger was treated with a formulation containing all of the ingredients of 5A-5G, in accordance with an embodiment of the invention. For each of 5A-5I, the finger was rubbed gently (streaked) onto an agar plate producing the letter ‘Z’, and the agar plates were each quickly placed in an incubator at 38 C. Photographs were taken at 24 hour intervals. The images shown in 5A-5I were taken 96 hours (4 days) after streaking was completed. After 96 hours, it is clear that the untreated finger showed the highest contamination (bacteria spread all over the plate). Each ingredient showed different levels of antimicrobial properties, with geranium oil being the most effective, and sandalwood seed oil being the least effective. The finger treated with an inventive composition that included all ingredients of 5A-5G resulted in no growth, as can be seen in 51. Several controls experiments were performed: each ingredient was deposited onto an agar plate and placed in an incubator at 38° C. for 3 days, but they didn't produce any growth. This demonstrates that the bacterial growth came from the finger and not from the ingredients.

FIG. 6A-6H illustrate, in accordance with various embodiments of the invention, images demonstrating the degree of penetration of water-based dye onto hands. 6A) A hand treated with a composition (“combined formulation”) that includes sea buckthorn seed oil, borage seed oil and sandalwood nut oil. A drop of pink food dye was then applied to the hand. 6B) The same hand as in 6A after wiping the dye away with a paper towel. 6C) The hand treated in 6A, then wiped with a paper towel in 6B, and subsequently washed with water and soap. 6D) A hand treated with a commercially available oil-based moisturizer, and then a drop of pink food dye was applied to the hand. 6E) the same hand in 6D after wiping the dye with a paper towel in the same manner as the hand treated in 6B. 6F) A hand first treated with a commercially available water-based moisturizer, then pink food dye was applied to the hand, and finally the hand was wiped with a paper towel. 6G) A hand treated with a commercially available water-based moisturizer, then pink food dye was applied, and finally the hand was washed with soap and water. H) A hand treated with the combined formulation described in 6A. A drop of pink food dye was applied to the hand 8 hours after the combined formulation was applied, and then the hand was wiped with a paper towel. The combined formulation significantly prevented penetration of the dye even 8 hours after the combined formulation was applied. In the experiments in FIGS. 6A-6H described above, in each case the hand was first washed with soap and water and then about 1 minute after application of each product (8 hours for the experiment shown in 6H), a drop of pink food dye was deposited in the center of the hand. Immediately after depositing the drop, the dye was spread quickly with one circular motion around the skin and then immediately removed using a paper towel by scrubbing the skin vigorously. A control experiment was performed on an untreated clean hand. These experiments demonstrated that oil-based products produced the highest level of protection by preventing the dye from penetrating the skin, because, using a minimum amount of water, it was possible to remove the dye completely. Although the effect of the inventive composition was similar to the commercially available oil-based product, the inventive composition showed a visibly superior hydrophobic effect, and the commercially available oil-based product was significantly greasier, whereas the inventive composition was completely invisible to the eye and to touch. A water-based commercially available product produced a less favorable result than an untreated hand.

FIG. 7A-7C illustrate, in accordance with various embodiments of the invention, the healing of contact dermatitis through the use of an inventive composition containing sandalwood nut oil, sea buckthorn seed oil, and red raspberry seed oil. FIG. 7A shows the subject's hand prior to daily treatment application of the inventive composition. FIG. 7B shows significant improvement of the subject's hand after 3 days of daily application of the inventive composition. FIG. 7C shows the subject's hand after 9 days of daily application of the inventive composition.

### DETAILED DESCRIPTION OF THE INVENTION

Unless defined otherwise, technical and scientific terms used herein have the same meaning as commonly understood by one of ordinary skill in the art to which this invention belongs. Bolognia et al., Dermatology (2012); Barel et al., Handbook of Cosmetic Science and Technology, (2014); and Smith, March's Advanced Organic Chemistry Reactions, Mechanisms and Structure 7^{th} ed., (2013); provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

Linolenic acid is a type of fatty acid, referring to either of two octadecatrienoic acids (i.e. with an 18-carbon chain and three double bonds, which are found in the cis configuration), or a mixture of the two. The two octadecatrienoic acids are alpha linolenic acid and gamma linolenic acid.

Linoleic acid (LA), a carboxylic acid, is a polyunsaturated omega-6 fatty acid, an 18-carbon chain with two double bonds in cis configuration. A shorthand notation includes “18:2 (n-6)” or “18:2 cis-9, 12.”

As used herein the terms “composition(s)” and “formulation(s)” have the same meaning.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or skin condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, preventing or repairing damage to skin from any source (e.g., electromagnetic radiation, burns, scalds, acne, weather conditions, pathogens, genetic defect(s), physical trauma, chemical trauma, disease (e.g. diabetes) and combinations thereof), improving the softness of skin, improving the tone of skin, protecting and healing skin that has been recently tattooed, reducing the appearance of wrinkles in skin, and relieving dryness. With respect to disease conditions, beneficial results may include lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy.

“Diseases,” “conditions” and “disease conditions,” as used herein may include, but are in no way limited to skin diseases or conditions such as acne, cellulitis, skin dryness, eczema, atopic dermatitis, contact dermatitis, seborrheic dermatitis, cradle cap, nummular dermatitis, stasis dermatitis, perioral dermatitis (muzzle rash) dermatitis herpetiformis, echtima, folliculitis, impetigo, keratosis, actinic (solar) keratosis, keratosis pilaris, keratosis follicularis, hyperkeratosis, photoallergy, skin conditions associated with diabetes, psoriasis, HSV1 and HSV2, chicken pox, herpes zoster, measles, rubella, stress, tattoo removal, burns, scars, dark spots, depigmentation, vitiligo, combinations thereof and the like.

As used herein, the term “administering,” refers to the placement of an agent, composition, or formulation as disclosed herein onto a subject by a method or route which results in at least partial localization of the agents or composition at a desired site. “Route of administration” may refer to any administration pathway known in the art, including but not limited to topical administration. Via the topical route, the agent or composition can be in the form of oil, skin scrub (i.e., formulation containing particulate matter that interacts with the skin), aerosol, droplet spray, lotion, cream, gel, ointment, suspensions, mousses, solutions or emulsions.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., dry skin or any other condition described herein) or one or more complications related to the condition (e.g., psoriasis), and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. For example, a subject can be one who exhibits one or more symptoms for a condition or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

### Skin Care Compositions

With the foregoing background in mind, in various embodiments, the invention teaches a skin care composition (also described herein as “formulation”) that includes one or more “protecting oils” that include ximenynic acid, and one or more oils that include linolenic acid (either alpha linolenic acid or gamma linolenic acid; or a mixture of both), and/or one or more oils that include linoleic acid.

In some embodiments, the one or more oils of the skin care formulation that include ximenynic acid may include, but are in no way limited to, an oil obtained from Ximenia or from Santalum sp. In some embodiments, the oil is obtained from a plant in the Santalaceae family. In some embodiments, the oil may be obtained from Ximenia Americana (also known as Mountain Plum or Manzanella or Cagalera or manzana del diablo or Tallow wood) or Ximenia Africana or Santalum spicatum or Santalum acuminatum (also known as Desert quandong or Sandalwood or Katunga or Burn-burn or Mangatais) or Santalum album. In certain embodiments one or more of the oils containing ximenynic acid are obtained from the fruit kernel of Blunt Sandalwood (Santalum obtusifolium) or the nut of an Australian Sandalwood (Santalum spicatum). In certain embodiments, one or more oils containing ximenynic acid are obtained from seed oil of other plants in the Santalaceae family including, but in no way limited to, the Native Cherry (Exocarpos cupressiformis).

In some embodiments, the one or more oils of the skin care formulation that include linolenic acid may include, but are in no way limited to, an oil obtained from evening primrose (Oenothera biennis), sea buckthorn seed, blackcurrant seed, borage seed, hemp seed, oats, barley, or spirulina.

In certain embodiments, the one or more oils of the skin care formulation that include linoleic acid may include but are in no way limited to salicornia oil, safflower oil, red raspberry seed oil, evening primrose oil, poppy seed oil, sunflower oil, barbary fig seed oil, hemp oil, corn oil, cotton seed oil, soybean oil, walnut oil, sesame oil, rice bran oil, argan oil, pistachio oil, peach pit oil, almond oil, canola oil, linseed oil, olive oil, palm oil, and coconut oil.

In various embodiments, one or more of the protecting oils described above are present in the skin care formulation in an amount ranging from about 1-99%, 10-90%, 20-80%, 30-70%, 40-60%, or 50-55% by weight. In some embodiments, one or more protecting oils that include ximenynic acid are present in the skin care formulation in an amount ranging from about 1-40%, 5-35%, 10-30%, or 15-25% by weight. In some embodiments, when oil extracted from Santalum album is one of the one or more oils in the formulation, it is present in an amount of about 12%, 13%, 14%, 15%, 16%, 17%, 18%, 19%, or 20% by weight. In some embodiments, when an oil extracted from a Santalum obtusifolium fruit kernel is one of the one or more oils in the formulation, it is present in an amount of about 18%, 19%, 20%, 21%, 22%, 23%, 24%, or 25% by weight. In certain embodiments, when an oil extracted from an Australian sandalwood (Santalum spicatum) nut is one of the one or more oils in the formulation, it is present in an amount of about 5%, 6%, 7%, 8%, 9%, 10%, 11%, 12%, 13%, 14%, or 15% by weight. In some embodiments, when present in the skin care formulation, one or more protecting oils that include linolenic acid are present in an amount of about 1-80%, 5-75%, 10-70%, 15-65%, 20-60%, 25-55%, 30-50%, or 35-45% by weight. In some embodiments, when oil obtained from evening primrose (Oenothera biennis) is present in the formulation, it is present in an amount of about 15%, 16%, 17%, 18%, 19%, 20%, 21%, 22%, 23%, 24%, 25%, 26%, 27%, or 28% by weight. In other embodiments, when oil obtained from evening primrose is present in the formulation, it is present in an amount of about 50%, 51%, 52%, 53%, 54%, 55%, 56%, or 57% by weight. In some embodiments, when oil obtained from sea buckthorn seed is present in the formulation, it is present in an amount of about 40%, 41%, 42%, 43%, 44%, 45%, 46%, 47%, 48%, 49%, 50%, 51%, 52%, 53%, 54%, 55%, 56%, 57%, 58%, 59%, or 60% by weight. In other embodiments, when oil obtained from sea buckthorn seed is present in the formulation, it is present in an amount of about 1%, 2%, 3%, 4%, 5%, 6%, 7%, 8%, 9%, 10%, 11%, 12%, 13%, 14%, or 15% by weight. In some embodiments, when oil obtained from blackcurrant borage seed is present in the formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight. In certain embodiments, when oil obtained from hemp seed is present in the formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight. In some embodiments, when present in the skin care formulation, one or more protecting oils that include linoleic acid are present in an amount of about 1-50%, 10-40%, or 20-30% by weight. In some embodiments, when present in the skin care formulation, salicornia oil is present in an amount of about 30%, 31%, 32%, 33%, 34%, 35%, 36%, 37%, 38%, 39%, 40%, 41%, 42%, 43%, 44%, 45%, 46%, or 47% by weight. In certain embodiments, when present in the skin care formulation, safflower oil is present in an amount of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% by weight. In certain embodiments, when present in the skin care formulation, red raspberry seed oil is present in an amount of about 20%, 21%, 22%, 23%, 24%, 25%, 26%, 27%, 28%, 29%, 30%, 31%, 32%, 33%, 34%, 35%, 36%, 37%, 38%, 39%, or 40% by weight. In other embodiments, when present in the skin care formulation, red raspberry seed oil is present in an amount of .5%, 1%, 1.5%, 2%, 2.5%, 3%, 3.5%, 4%, 4.5%, 5%, 5.5%, 6%, 6.5%, 7%, 7.5%, or 8% by weight. In some embodiments, when present in the skin care formulation, evening primrose oil is present in an amount of about 5%, 5.5%, 6%, 6.5%, 7%, 7.5%, 8%, or 8.5% by weight. In certain embodiments, when present in the skin care formulation, poppy seed oil is present in an amount of about 8%, 9%, 10%, 11%, 12%, 13%, 14%, 15%, 16%, or 17% by weight. In some embodiments, when present in the skin care formulation, sunflower oil is present in an amount of about 40%, 41%, 42%, 43%, 44%, 45%, 46%, 47%, 48%, or 49% by weight. In some embodiments, when present in the skin care formulation, coconut oil is present in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight. In some embodiments, when present in the skin care formulation, argan oil is present in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight.

In some embodiments, in addition to the one or more protecting oils, the skin care formulation also includes one or more base oils. In some embodiments, one or more of the base oils are present in the formulation in an amount of about 10-60%, 20-50%, or 30-40% by weight. In some embodiments, the base oil may include, but is in no way limited to, jojoba oil, safflower oil, sunflower oil, olive oil, or combinations thereof. In certain embodiments, when present in the skin care formulation, jojoba oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin care formulation, safflower oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin care formulation, sunflower oil is present in an amount of about 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin care formulation, olive oil is present in an amount of about 21%, 22%, 23%, 24%, 25%, 26%, 27%, 28%, 29%, 30%, 31%, 32%, 33%, 34%, 35%, 36%, 37%, 38%, 39%, or 40% by weight.

In certain embodiments, the skin care formulation includes one or more protecting oils and one or more base oils and/or one or more “nutrient oils.” In some embodiments, one or more of the nutrient oils may be obtained from pomegranate seed, pumpkin seed, hazelnut, maracuja seed, blueberry seed, evening primrose, almond, rosehip, or apricot kernel. In certain embodiments, one or more of the nutrient oils are present in the formulation in an amount of about 0.5%-10%, 3.5-7%, or 5-6% by weight. In some embodiments, when present in the skin care formulation, pomegranate seed oil is present in an amount of about 0.5%, 1%, 1.5%, 2%, 2.5%, 3%, 3.5%, 4%, 4.5%, 5%, 5.5%, 6%, 6.5%, 7%, 7.5%, or 8% by weight. In some embodiments, when present in the skin care formulation, pumpkin seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin care formulation, hazelnut oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, or 8.5% by weight. In some embodiments, when present in the skin care formulation, pumpkin seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin care formulation, maracuja seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin care formulation, blueberry seed oil is present in an amount of about 0.5%, 1%, 1.5%, 2%, 2.5%, 3%, 3.5%, 4%, 4.5%, 5%, 5.5%, 6%, 6.5%, 7%, or 7.5% by weight. In some embodiments, when present in the skin care formulation, evening primrose oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin care formulation, almond oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin care formulation, rosehip oil is present in an amount of about 0.5%, 1%, 1.5%, 2%, 2.5%, 3%, 3.5%, 4%, 4.5%, 5%, 5.5%, or 6% by weight. In some embodiments, when present in the skin care formulation, apricot kernel oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, or 9% by weight.

In certain embodiments, the skin care formulation includes one or more protecting oils and one or more base oils and/or one or more nutrient oils and/or one or more “essential oils.” In certain embodiments, one or more of the essential oils may contain one or more of limonene, linalool, geraniol, and citral. In some embodiments, the one or more oils that include limonene are obtained from peels of citrus plants (e.g., grapefruit, orange, lemon, tangerine, lime, pomelo, bergamot and the like). In some embodiments, the one or more oils that include linalool may include but are in no way limited to an oil obtained from coriander (Coriandrum sativum L. family Apiaceae) seed, palmarosa [Cymbopogon martinii var martinii (Roxb.) Wats., family Poaceae], sweet orange (Citrus sinensis Osbeck, family Rutaceae) flowers, lavender (Lavandula officinalis Chaix, family Lamiaceae), bay laurel (Laurus nobilis, family Lauraceae), sweet basil (Ocimum basilicum, family Lamiaceae), and bergamot. In some embodiments, the one or more oils that include geraniol may include but are in no way limited to rose oil, palmarosa oil, and citronella oil (Java type), geranium oil, and lemon oil. In some embodiments, the one or more oils that include citral may include but are in no way limited to an oil obtained from lemon myrtle, Litsea citrata, Litsea cubeba, lemongrass, lemon tea-tree, Ocimum gratissimum, Indera citriodora, Calypranthes parriculata, petitgrain, lemon verbena, ironbark, lemon balm, lime peel, lemon peel, and orange peel.

In some embodiments, one or more of the essential oils may include, but are in no way limited to, bergamot essential oil, grapefruit essential oil, citronella essential oil, and lemon myrtle essential oil. In certain embodiments, one or more of the essential oils are present in the skin scrub formulation in an amount of about 0.5%-11%, 3.5-8%, or 5-6% by weight. In some embodiments, when bergamot essential oil is present in the skin care formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In certain embodiments, when grapefruit essential oil is present in the skin care formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight.

In certain embodiments, the invention teaches a composition that can be applied to the skin of a mammal, including a human mammal, which includes sea buckthorn seed oil and sandalwood nut oil. In some embodiments, the composition further includes red raspberry seed oil. In certain embodiments the composition includes 35-65% by weight Sea buckthorn seed oil and 5-20% by weight Sandalwood nut oil. In some embodiments, the composition further includes 20-40% by weight red raspberry seed oil.

In some embodiments, when the composition includes Sea buckthorn seed oil and sandalwood nut oil, the composition includes 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% by weight Sea buckthorn seed oil and 5%, 6%, 7%, 8%, 9%, 10%, 11%, 12%, 13%, 14%, 15%, 16%, 17%, 18%, 19%, or 20% by weight sandalwood nut oil. In certain embodiments, the composition further includes 20%, 21%, 22%, 23%, 24%, 25%, 26%, 27%, 28%, 29%, 30%, 31%, 32%, 33%, 34%, 35%, 36%, 37%, 38%, 39%, or 40% by weight red raspberry seed oil.

In some embodiments, the invention teaches a composition that can be applied to the skin of a mammal, including a human mammal, wherein the composition includes 50-85% by weight safflower oil and 15-35% by weight cranberry seed oil.

In some embodiments, when the composition includes safflower oil and cranberry seed oil, the composition includes 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight safflower oil and 15%, 16%, 17%, 18%, 19%, 20%, 21%, 22%, 23%, 24%, 25%, 26%, 27%, 28%, 29%, 30%, 31%, 32%, 33%, 34%, or 35% by weight cranberry seed oil.

In some embodiments, the invention teaches a composition that can be applied to the skin of a mammal, including a human mammal, wherein the composition includes 40-70% by weight safflower oil and 8-25% by weight sandalwood nut oil. In some embodiments, the composition further includes 10-20% by weight maracuja seed oil. In some embodiments, the composition still further includes 3-12% by weight Neroli oil.

In some embodiments, when the composition includes safflower oil and sandalwood nut oil, the composition includes 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight safflower oil and 8%, 9%, 10%, 11%, 12%, 13%, 14%, 15%, 16%, 17%, 18%, 19%, 20%, 21%, 22%, 23%, 24%, 25% by weight sandalwood nut oil. In some embodiments, the composition further includes 10%, 11%, 12%, 13%, 14%, 15%, 16%, 17%, 18%, 19%, or 20% by weight maracuja seed oil. In some embodiments, the composition still further includes 3%, 4%, 5%, 6%, 7%, 8%, 9%, 10%, 11% or 12% by weight Neroli oil.

In some embodiments, a composition is applied to the skin of a mammal, including a human mammal, wherein the composition includes 20-40% by weight sandalwood nut oil and 40-65% by weight safflower oil. In some embodiments, the invention further includes 1-10% rose otto oil.

In some embodiments, when the composition includes sandalwood nut oil and safflower oil, the composition includes 20%, 21%, 22%, 23%, 24%, 25%, 26%, 27%, 28%, 29%, 30%, 31%, 32%, 33%, 34%, 35%, 36%, 37%, 38%, 39%, or 40% by weight sandalwood nut oil and 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% by weight safflower oil. In some embodiments, the composition further includes 1%, 2%, 3%, 4%, 5%, 6%, 7%, 8%, 9% or 10% by weight rose otto oil.

In certain embodiments, a composition is applied to the skin of a mammal, including a human mammal, wherein the composition includes 5-15% by weight sea buckthorn seed oil and 10-30% by weight sandalwood nut oil. In some embodiments, the composition further includes 5-25% by weight borage seed oil. In some embodiments, the composition still further includes 15-40% by weight red raspberry seed oil.

In some embodiments, when the composition includes sea buckthorn seed oil and sandalwood nut oil, the composition includes 5%, 6%, 7%, 8%, 9%, 10%, 11%, 12%, 13%, 14%, or 15% by weight sea buckthorn seed oil and 10%, 11%, 12%, 13%, 14%, 15%, 16%, 17%, 18%, 19%, 20%, 21%, 22%, 23%, 24%, 25%, 26%, 27%, 28%, 29%, or 30% by weight sandalwood nut oil. In some embodiments, the composition further includes 5%, 6%, 7%, 8%, 9%, 10%, 11%, 12%, 13%, 14%, 15%, 16%, 17%, 18%, 19%, 20%, 21%, 22%, 23%, 24%, or 25% by weight borage seed oil. In some embodiments, the composition still further includes 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight red raspberry seed oil.

In certain embodiments, a composition is applied to the skin of a mammal, including a human mammal, wherein the composition includes 15-40% by weight ground coffee and 10-30% by weight safflower oil. In some embodiments, the composition further includes 3-10% by weight salt. In some embodiments, the composition further includes 25-45% sugar (e.g., powdered sugar or granulated sugar). In some embodiments, the salt is Himalayan sea salt. In some embodiments, as a substitute for safflower oil, blackberry seed oil 15-33% by weight, or hemp oil 11-28% by weight, or jojoba 6-25% by weight, or their combination is included in the formulation.

In some embodiments, when the composition includes ground coffee, salt, and sugar, the composition includes 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight ground coffee and 3%, 4%, 5%, 6%, 7%, 8%, 9%, or 10% by weight salt and 25%, 26%, 27%, 28%, 29%, 30%, 31%, 32%, 33%, 34%, 35%, 36%, 37%, 38%, 39%, 40%, 41%, 42%, 43%, 44%, or 45% by weight sugar (e.g., powdered sugar or granulated sugar). In some embodiments, the composition further includes 10%, 11%, 12%, 13%, 14%, 15%, 16%, 17%, 18%, 19%, 20%, 21%, 22%, 23%, 24%, 25%, 26%, 27%, 28%, 29%, or 30% by weight safflower oil, 3%, 4%, 5%, 6%, 7%, 8%, 9%, 10%, 11% by weight jojoba oil, or 1%, 2%, 3%, 4%, 5%, 6%, 7%, 8% by weight sandalwood seed oil, or 2%, 3%, 4%, 5%, 6%, 7%, 8%, 9%, 10%, 11%, 12%, 13%, 14%, 15%, 15%, 16%, 17% by weight of blackberry seed oil.

Certain non-limiting examples of skin care compositions/formulations (AV1-AV10) of various embodiments of the present disclosure are set forth below in Table 1 (all values are % weight).

TABLE 1

| Ingredient | AV1 | AV2 | AV3 | AV4 | AV5 | AV6 | AV7 | AV8 | AV9 | AV10 |
|---|---|---|---|---|---|---|---|---|---|---|
| Sea | 50 | 1 | 2 | 4 | 22 | 9 | 1 | 3 | 25 | 11 |
| Buckthorn | | | | | | | | | | |
| seed oil | | | | | | | | | | |
| Red | 30 | 3 | 2 | 1 | | 20 | | | 25 | |
| Raspberry | | | | | | | | | | |
| seed oil | | | | | | | | | | |
| Sandalwood | 10 | | | | 33 | | 25 | | 11 | 30- |
| nut oil | | | | | | | | | | |
| Pomegranate | 2 | 3 | 5 | 2 | 2.10 | 7 | | 4 | | |
| seed oil | | | | | | | | | | |
| Borage seed | 2 | 3 | 1 | 1 | | | | 2 | | 2 |
| oil | | | | | | | | | | |
| Pumpkin | 2 | 3 | | | | | | | 1 | |
| seed oil | | | | | | | | | | |
| Hazelnut oil | 1 | 3 | | 1 | | 4 | | 1 | 2 | 3 |
| Maracuja | 1 | 3 | | | | | | | 1 | 4 |
| seed oil | | | | | | | | | | |
| Rosehip oil | 1 | 1 | | 1 | | 3 | | | 1 | |
| Coconut oil | | | 10 | 20 | 9 | 25 | 14 | 30 | 4 | 25 |
| Blueberry | | | | | | 10 | | | | 8 |
| seed oil | | | | | | | | | | |
| Apricot | | | | | | 8 | | | 5 | |
| kernel oil | | | | | | | | | | |
| Argan oil | | 10 | 20 | 20 | | 14 | | 20 | 25 | 17 |
| Avocado oil | | | | | | | 6 | | | |
| Jojoba oil | | 20 | 45 | 35 | 9 | | 40 | 18 | | |
| Safflower oil | | 50 | 15 | 15 | 23 | | 14 | 22 | | |
| Bergamot | 0.3 | | | | | | | | | |
| essential oil | | | | | | | | | | |
| Grapefruit | 0.4 | | | | | | | | | |
| essential oil | | | | | | | | | | |
| Frankicense | 0.3 | | | | | | | | | |
| essential oil | | | | | | | | | | |
| Salt | | | | | 1.9 | | | | | |
| TOTAL % | 100 | 100 | 100 | 100 | 100 | 100 | 100 | 100 | 100 | 100 |

In some embodiments, one or more oils of the skin care formulations described herein are obtained by cold pressing the nut, seed, or any other portion of a plant described herein and decanting, as described in Hanna, M. A. et al, ‘Expression of oil from oilseeds—A review’ J Agric. Eng. Res., 1998, 28 (6) 495-583, which is incorporated herein by reference in its entirety. In some embodiments, oils are extracted from the nut, seed, or any other portion of a plant described herein through the use of supercritical CO2, as described in Ibanez, E. et al, ‘Sub—and supercritical fluid extraction of functional ingredients from different natural sources: Plants, food-by-products, algae and microalgae: A review’, 2006, 98(1), 136-148 which is incorporated herein by reference in its entirety.

Certain formulations described herein which contain ximenynic acid and linolenic acid and/or linoleic acid have surprisingly and unexpectedly strong antioxidant, insect repellent, and ultraviolet radiation protection properties, while also absorbing rapidly into the skin and leaving little to no perceptible oily, greasy, or sticky residue. These formulations include, but are in no way limited to those that include sandalwood nut oil and sea buckthorn seed oil. Formulations that further include red raspberry seed oil are especially effective as skin care formulations with the aforementioned attributes. Daily application of these formulations resulted in a reduced appearance of fine lines and wrinkles on the faces and hands of study subjects, and has been shown to provide strong antioxidant protection along with protection against ultraviolet light. The formulations described herein have also been shown to improve symptoms of psoriasis, dermatitis, and eczema in human subjects, as described in greater detail in the examples set forth herein.

The skin care compositions according to various embodiments described herein may be delivered topically in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic composition and the physiological condition of the subject (including, but not limited to, age, sex, disease type and stage, general physical condition, responsiveness to a given dosage, and type of composition).

### Methods of Skin Care Treatment

In various embodiments, the present invention provides a method for treating a subject's skin, including skin on any portion of the subject's body. In certain embodiments, the method includes applying a formulation described herein to the subject's skin. In some embodiments, the method includes applying a formulation described herein to the subject's scalp. In certain embodiments, the method includes applying a formulation described herein to the subject's lips. In some embodiments, the method includes applying a formulation described herein to a subject's cuticle. In certain embodiments, the method includes applying a formulation described herein to a subject's foot. In some embodiments, the method includes applying a formulation described herein to a subject's face. In certain embodiments, the method includes applying a formulation described herein to a subject's face, and then applying makeup to the subject's face. In certain embodiments, a formulation described herein is applied to skin before or after shaving hair on the skin.

In some embodiments, the invention teaches a method for treating a subject's skin that includes applying a skincare formulation that includes sandalwood nut oil and sea buckthorn seed oil to the subject's skin. In some embodiments, the skincare formulation applied to the subject's skin further includes red raspberry seed oil. In some embodiments, the method includes applying formulation AV1 of Table 1 onto a subject's skin. In some embodiments, the method includes applying formulation AV2 of Table 1 onto a subject's skin. In some embodiments, the method includes applying formulation AV3 of Table 1 onto a subject's skin. In some embodiments, the method includes applying formulation AV4 of Table 1 onto a subject's skin. In some embodiments, the method includes applying formulation AV5 of Table 1 onto a subject's skin. In some embodiments, the method includes applying formulation AV6 of Table 1 onto a subject's skin. In some embodiments, the method includes applying formulation AV7 of Table 1 onto a subject's skin. In some embodiments, the method includes applying formulation AV8 of Table 1 onto a subject's skin. In some embodiments, the method includes applying formulation AV9 of Table 1 onto a subject's skin. In some embodiments, the method includes applying formulation AV10 of Table 1 onto a subject's skin.

In some embodiments, topical application of the formulations described herein is accomplished by hand. In certain embodiments, topical application of the formulations described herein is accomplished with one or more of a dropper, brush, sponge, wipe, syringe, straw, hose.

In certain embodiments, any of the formulations described herein may be incorporated into an absorbent material, including but in no way limited to, a sponge, a wipe, a tissue, a pad, a paper, a cloth, a towel, a cotton ball, a glove, a gauze, a band-aid, a patch.

In some embodiments, a composition as disclosed herein may be administered at the prevention stage (i.e., when the subject has not developed any of the conditions described herein, but is likely to or in the process of developing a condition described herein (e.g. wrinkles, dry skin, psoriasis, dermatitis, cellulitis, eczema, etc.)). In other embodiments, a composition as disclosed herein may be administered at the treatment stage (e.g., when the subject has already developed wrinkles, dry skin, psoriasis, dermatitis, cellulitis, eczema, etc.). In other embodiments, a composition as disclosed herein may be administered at the maintenance stage (e.g., when the subject has been successfully treated for wrinkles, dry skin, psoriasis, dermatitis, cellulitis, eczema, etc.). Administering the skincare formulations described herein to a subject's skin at the prevention stage, treatment stage, or maintenance stage is within the scope of the methods of various embodiments of the invention.

In certain embodiments, the invention teaches a method that includes applying a skincare formulation described herein to a subject who has one or more of wrinkles, dry skin, psoriasis, dermatitis, cellulitis, eczema. In some embodiments, the method includes applying a skincare formulation that includes sandalwood nut oil and sea buckthorn seed oil to a subject's skin. In some embodiments, the formulation applied to the subject's skin further includes red raspberry seed oil.

When in oil form, formulations described herein are applied in sufficient quantities to coat the skin to be treated. In various embodiments, any of the formulations described herein may be applied to a subject's skin 1-20 or more times per day. In various embodiments, any of the formulations described herein may be applied to a subject's skin 1, 2, 3, 4, 5, 6, 7, 8, 9, 10 or more times per day.

Ingredients of the same or two or more compositions of the present invention can be applied in separate steps or simultaneously depending on the type of container used. Merely by way of example, a formulation that includes sea buckthorn seed oil (e.g. in a concentration within a range described above, or as the only ingredient) and/or raspberry seed oil (e.g. in a concentration within a range described above, or as the only ingredient) may be applied in a first step, and then a formulation that includes sandalwood nut oil (e.g. in a concentration within a range described above, or as the only ingredient) may be applied in a subsequent step (e.g. in a second step). The two or more formulations can be dispensed from physically separate packages or from a unitary package with chambers. Examples of packages include, but are not limited to a pouch inside of a pouch, or a dual bladder system inside of a can. The components of either type of packages can be applied simultaneously or substantially simultaneously to the skin, where they commingle. In some embodiments, in the first step, one formulation is applied to the skin and in the second step, the other formulation is applied over the first component within a period of 1 second or less to 1 hour or more. The components are, thus, applied substantially simultaneously such that commingling occurs when the second component is applied on top of the first component.

### Skin Scrub Compositions

Unfortunately, most commercially available skin scrubs and cleansers lack a meaningful moisturizing effect and/or are harsh on skin, and therefore can only be used occasionally (e.g., 1-2 times per week). Additionally, many of these products are not “natural,” as they include ingredients that have been chemically processed and/or synthesized and are often damaging to the environment. Skin care compositions/formulations, including skin scrubs and cleansers, that improve the condition of skin while not being harsh or leaving a greasy or sticky residue. Skin care formulations that can achieve these results while also providing sun protection and/or antimicrobial and/or antiaging properties are especially needed.

With the foregoing background in mind, in various embodiments, the invention teaches a skin care composition (also described herein as a “formulation” or “skin formulation”) that includes one or more “protecting oils” that include ximenynic acid, one or more “scrubbing ingredient” containing particles suitable for mechanically interacting with skin, and/or one or more oils that include linolenic acid, and/or one or more oils that include linoleic acid. Skin care formulations described herein include, but are in no way limited to, formulations intended to “scrub” and/or “cleanse” skin. The skin care formulations described herein that include a scrubbing ingredient are also referred to as “skin scrubs.”

In some embodiments, the one or more oils of the skin scrub formulation that include ximenynic acid may include, but are in no way limited to, an oil obtained from Ximenia or from Santalum sp. In some embodiments, the oil is obtained from a plant in the Santalaceae family. In some embodiments, the oil may be obtained from Ximenia Americana (also known as Mountain Plum or Manzanella or Cagalera or manzana del diablo or Tallow wood) or Ximenia Africana or Santalum spicatum or Santalum acuminatum (also known as Desert quandong or Sandalwood or Katunga or Burn-burn or Mangatais) or Santalum album. In certain embodiments one or more of the oils containing ximenynic acid are obtained from the fruit kernel of Blunt Sandalwood (Santalum obtusifolium) or the nut of an Australian Sandalwood (Santalum spicatum). In certain embodiments, one or more oils containing ximenynic acid are obtained from seed oil of other plants in the Santalaceae family including, but in no way limited to, the Native Cherry (Exocarpos cupressiformis).

In some embodiments, one or more scrubbing ingredient of the skin scrub formulation may include, but is in no way limited to, one or more of coffee bean particles (e.g., resulting from crushing, grinding, pulverizing, smashing, combinations thereof and the like), sugar (e.g., granulated, powdered, or otherwise processed), salt, almond flour, amaranth flour, barley flour, buckwheat flour, corn flour, flaxseed flour or meal, oat flour, peanut flour, potato flour, rice flour (e.g., brown or white), rye flour, soy flour, cassava flour, chickpea flour, chapatti flour, dal flour, fufu flour, kamut flour, millet flour, teff flour, fruit seed flour (e.g. seeds of fruits), raspberry seed flour, pomegranate seed flour, blackberry seed flour, orange seed flour, Davidson Plum (Davidsonia jerseyana) fruit powder extract, Desert Lime (Citrus glauca) a fruit powder extract, Emu Apple (Kunzea pomifera), Finger Lime (Citrus australasica) fruit powder extract, Kakadu Plum (Terminalia ferdinandiana) fruit powder extract, Lemon Aspen (Acronychia acidula) fruit powder extract, Quandong (Santalum acuminatum) fruit powder extract, Lilli Pilli/Riberry (Syzygium leuhmannii) fruit powder extract, Mountain Pepper Leaf (Tasmannia lanceolate) leaf powder extract, avocado pit particles (e.g., resulting from crushing, grinding, pulverizing smashing, combinations thereof and the like), watermelon seed particles (e.g., resulting from crushing, grinding, pulverizing smashing, combinations thereof and the like), nut shell particles (e.g. one or more of walnut, pistachio, and brazil nut particles resulting from crushing, grinding, pulverizing smashing, combinations thereof and the like), combinations thereof and the like. In certain embodiments, the sizes of the individual particles of one or more scrubbing ingredients ranges from 0.001-0.8 mm, although smaller and larger particles may be used. In certain embodiments, the sizes of the individual particles of one or more scrubbing ingredients ranges from 0.05-0.6 mm, 0.07-0.4 mm, 0.09-0.2 mm, or 0.1-0.1 mm. Merely by way of non-limiting example, when salt is used as one of the scrubbing ingredients, it may be in a size range of 0.05 to 0.5 mm. In certain non-limiting embodiments, salt may be found in the formulations described herein in a size range of 0.1 to 0.75 mm. In certain non-limiting embodiments, sugar may be found in the formulations described herein in a size range from 0.01 to 0.5 mm. In certain non-limiting embodiments, any seed flour described herein may be present in the formulations described herein in a size range of 0.001 to 0.1. In some embodiments, one or more scrubbing ingredients are present in the skin care formulations in an amount ranging from about 1-40%, 5-35%, 10-30%, or 15-25% by weight.

In certain embodiments, in addition to the scrubbing ingredient(s), the skin scrub compositions may be water-based. Such compositions can be referred to as water-based primers. In some embodiments, the skin scrub compositions/water-based primers include water and 0.1-20% by weight of one or more ingredients set forth in Table 2.

TABLE 2

| Ingredient | % weight |
|---|---|
| Aniseed Myrtle (Backhousia anisate) Leaf Extract | 0.1-20% |
| Banksia (Banksia serrata) Flower and/or Leaf Extract | 0.1-20% |
| Blackwood Wattle (Acacia melanoxylon) Leaf Extract | 0.1-20% |
| Blue Cypress (Callitirs intratropica) Leaf Extract | 0.1-20% |
| Blue Cypress (Callitris intratropica) Wood Extract | 0.1-20% |
| Crown of Gold (Barkyla syringifolia) Flower Extract | 0.1-20% |
| Davidson Plum (Davidsonia jerseyana) Fruit Extract | 0.1-20% |
| Desert Lime (Citrus glauca) Pulp and/or Skin Extract | 0.1-20% |
| Emu Apple (Kunzea pomifera) Fruit Extract | 0.1-20% |
| Emu Bush (Eremophila longifolia) Leaf Extract | 0.1-20% |
| Finger Lime Caviar (Citrus australasica) Pulp Extract | 0.1-20% |
| Illawarra Flame Tree (Brachychiton acerifolius) Flower Extract | 0.1-20% |
| Kakadu Plum (Terminalia ferdinandiana) Fruit Extract | 0.1-20% |
| Kangaroo Apple (Solanum aviculare) Leaf Extract | 0.1-20% |
| Kangaroo Paw (Anigozanthos flavidus) Leaf and/or Flower Extract | 0.1-20% |
| Kapok Tree (Cochlospermum fraseri) Leaf Extract | 0.1-20% |
| Lemon Aspen (Acronychia acidula) Fruit Extract | 0.1-20% |
| Lemon Myrtle (Backhousia citriodora) Leaf Extract | 0.1-20% |
| Macadamia Leaf (Macadamia integrifolia) Leaf Extract | 0.1-20% |
| Lilli Pilli (Syzygium luehmannii) Leaf Extract | 0.1-20% |
| Manuka Honey (Leptosperumum scoparium) Honey Extract | 0.1-20% |
| Mountain Pepper (Tasmannia lanceolate) Berries Extract | 0.1-20% |
| Muntries (Kunzea pomifera) Fruit Extract | 0.1-20% |
| Native Orange Pearl (Malcura cochinchinensis) Leaf Extract | 0.1-20% |
| Native River Mint (Mentha australis) Leaf Extract | 0.1-20% |
| Native Seaweed (Ulva lactuca) Leaf Extract | 0.1-20% |
| Native Snowflower (Melaleuca alternifolia) Leaf Extract | 0.1-20% |
| Quandong (Santalum acuminatum) Fruit Extract | 0.1-20% |
| Red Mallotus (Mallotus philippinensis) Berries Extract | 0.1-20% |
| Riberries (Syzygium luehmannii) Berries Extract | 0.1-20% |
| Rosella (Hibiscus abelmoschus) Fruit Extract | 0.1-20% |
| Silky Oil Grass (Cymbopogan bombycinus) Grass Extract | 0.1-20% |
| Snake Vine (Hibbertia scanderns) Vine Extract | 0.1-20% |
| Strawberry Gum (Eucalyptus olida) Leaf Extract | 0.1-20% |
| Tasmanian Blue Gum (Eucalyptus globulus) Leaf Extract | 0.1-20% |
| Waratah (Telopea speciosissima) Flower Extract | 0.1-20% |
| White Cypress (Calitris glaucophylla) Leaf Extract | 0.1-20% |
| Wild Orange (Capparis mitchellii) Leaf Extract | 0.1-20% |
| Agrimony (Agrimonia eupatoria) Herb Extract | 0.1-20% |
| Arnica (Arnica montana) Flower Extract | 0.1-20% |
| Balm Mint (Melissa officinalis) Leaf Extract | 0.1-20% |
| Birch (Betula alba) Leaf Extract | 0.1-20% |
| Calendula (Calendula officinalis) Flower Extract | 0.1-20% |
| Chamomile (Matricaria chamomilla (reutita)) Flower Extract | 0.1-20% |
| Chilli/Capsicum (Capsicum annuum) Fruit Extract | 0.1-20% |
| Coltsfoot (Tussilago farfara) Leaf Extract | 0.1-20% |
| Comfrey (Symphytum officinale) Leaf Extract | 0.1-20% |
| Cucumber (Cucumis sativus) Fruit Extract | 0.1-20% |
| Echinacea (Echinacea purpurea) Herb Extract | 0.1-20% |
| Elderflower (Sambucus nigra) Herb Extract | 0.1-20% |
| Eyebright (Euphrasia officunalis) Herb Extract | 0.1-20% |
| Gentian (Gentiana lutea) Aerials Extract | 0.1-20% |
| Gingko (Ginkgo biloba) Aerials Extract | 0.1-20% |
| Ginseng (Eleutherococcus senticosus) Root Extract | 0.1-20% |
| Green Tea (Camellia sinensis) Leaf Extract | 0.1-20% |
| Hops (Humulus lupulus) Aerials Extract | 0.1-20% |
| Horse Chestnut (Aesculus hippocastanum) Nut Extract | 0.1-20% |
| Jiaogulan (Gynostemma pentaphyllum) Leaf Extract | 0.1-20% |
| Kelp (Fucus vesiculosus) Leaf Extract | 0.1-20% |
| Lavender (Lavendula angustifolia) Flower Extract | 0.1-20% |
| Licorice (Glycyrrhiza glabra) Root Extract | 0.1-20% |
| Marshmallow (Althaea officinalis) Leaf Extract | 0.1-20% |
| Milk Thistle (Silybum marianum) Seed Extract | 0.1-20% |
| Mugwort (Artemisia vulgaris) Herb Extract | 0.1-20% |
| Nettle (Urtica dioica) Herb Extract | 0.1-20% |
| Oakbark (Quercus alba) Bark Extract | 0.1-20% |
| Oatmeal (Avena sativa) Seed Extract | 0.1-20% |
| Olive Leaf (Olea europea) Leaf Extract | 0.1-20% |
| Parsley (Petroserlinum crispum) Leaf Extract | 0.1-20% |
| Restharrow (Ononis spinose) Aerials Extract | 0.1-20% |
| Rosehip (Rosa canina) Fruit Extract | 0.1-20% |
| Rosemary (Rosmarinus officinalis) Leaf Extract | 0.1-20% |
| Sage (Salvia officinalis) Leaf Extract | 0.1-20% |
| Saw Palmetto (Serenoa serrulata) Fruit Extract | 0.1-20% |
| Sea Fennel (Crithmum maritmum) Leaf Extract | 0.1-20% |
| Soapworth (Saponaria officinalis) Bark Extract | 0.1-20% |
| St John's Wort (Rosin Rose) (Hypericum perforatum) Aerials Extract | 0.1-20% |
| Thyme (Thymus vulgaris) Leaf Extract | 0.1-20% |
| Vanilla (Vanilla fragrans) Pod Extract | 0.1-20% |
| Willow Bark (Salix alba) Bark Extract | 0.1-20% |
| Willow Herb (Epilobium angustifolium) Leaf Extract | 0.1-20% |
| Witchhazel (Hammamalis virginiana) Leaf and/or Bark Extract | 0.1-20% |
| Wormwood (Arlemisia absinthium) Wood Extract | 0.1-20% |
| Yarrow (Achillea millefolium) Root Extract | 0.1-20% |
| Agapanthus (Agapanthus africanus) Flower Extract | 0.1-20% |
| Banksia (Banksia serrata) Flower Extract | 0.1-20% |
| Blue Butterfly (Delphinium grandiflorum) Flower Extract | 0.1-20% |
| Blue Lotus (Nymphaea caeulea) Flower Extract | 0.1-20% |
| Boronia (Boronia megastigma) Flower Extract | 0.1-20% |
| Bromeliad (Aechmea leucolepis) Flower Extract | 0.1-20% |
| Chrysanths (Chrysanthemum morifolium) Flower Extract | 0.1-20% |
| Cornflower (Centaurea cyanus) Flower Extract | 0.1-20% |
| Daphne (Daphne gnidium) Flower Extract | 0.1-20% |
| Flannel (Actinotus helianthi) Flower Extract | 0.1-20% |
| Frangipani (Plumeria alba) Flower Extract | 0.1-20% |
| Freesia (Freesia grandiflora) Flower Extract | 0.1-20% |
| Gardenia (Gardenia jasminoides) Flower Extract | 0.1-20% |
| Hyacinth (Hyacinthus orientalis) Flower Extract | 0.1-20% |
| Hydrangea (Hydrangea macrophylla) Flower Extract | 0.1-20% |
| Iris (Iris germanica) Flower Extract | 0.1-20% |
| Jacaranda (Jacaranda mimosaefolia) Flower Extract | 0.1-20% |
| Lily (Lilium candidum) Flower Extract | 0.1-20% |
| Orange Blossum (Murraya paniculata) Flower Extract | 0.1-20% |
| Red Tower Ginger (Costus comosus) Flower Extract | 0.1-20% |
| Waratah (Telopea speciosissima) Flower Extract | 0.1-20% |
| Avocado (Persea gratissimi) Leaf Extract | 0.1-20% |
| Banana (Musa paradisiaca) Fruit Extract | 0.1-20% |
| Beetroot (Beta vugaris) Vegetable Extract | 0.1-20% |
| Bitter Orange (Citrus aurantium amara) Fruit Extract | 0.1-20% |
| Blackberry (Ribus fruticosus) Fruit Extract | 0.1-20% |
| Blackcurrant (Ribes nigrum) Fruit Extract | 0.1-20% |
| Blueberry (Vaccinium corymbosum) Fruit Extract | 0.1-20% |
| Broccoli (Brassica oleracea Iralica) Vegetable Extract | 0.1-20% |
| Brussels Sprout (Brassica oleracea Gamifera) Vegetable Extract | 0.1-20% |
| Cherry (Prunus avium) Fruit Extract | 0.1-20% |
| Cherry (Prunus avium) Fruit Extract | 0.1-20% |
| Coffee Bean (Coffea robusta) Bean Extract | 0.1-20% |
| Cucumber (Cucumis sativus) Fruit Extract | 0.1-20% |
| Custard Apple (Annona reticulate) Fruit Extract | 0.1-20% |
| Dragonfruit (Hylocereus undatus) Fruit Extract | 0.1-20% |
| Kale (Brassica oleracea) Leaf Extract | 0.1-20% |
| Lime Caviar (Citrus aurantifolia) Fruit Pulp Extract | 0.1-20% |
| Mango (Mangifera indica) Frui Extract | 0.1-20% |
| Papaya (Carica) Fruit Extract | 0.1-20% |
| Passionfruit (Passiflora edulis) Fruit Extract | 0.1-20% |
| Paw Paw (Asimina triloba) Fruit Extract | 0.1-20% |
| Peach (Prunus persica) Fruit Extract | 0.1-20% |
| Pomegranate (Punica granatum) Fruit Extract | 0.1-20% |
| Portobello (Agaricus campestris) Fungi Extract | 0.1-20% |
| Shitake (Lentinula edodes) Fungi Extract | 0.1-20% |
| Watermelon (Citrullus lanatus) Fruit Extract | 0.1-20% |

Table 3 below demonstrates exemplary non-limiting ranges of concentrations of ingredients for formulations of water-based primers (WP1-WP4 represent the names of compositions with specific combinations of ingredients (listed in the left most column)) in accordance with various embodiments of the invention. All values listed in ranges are % weight

TABLE 3

| | Formulations (ranges listed are % weight) | | | |
|---|---|---|---|---|
| Ingredient | WP1 | WP2 | WP3 | WP4 |
| Kakadu Plum Terminalia | | 0.1-4 | 0.1-2 | 0.1-6 |
| ferdinandiana Extract Fruit | | | | |
| Lemon Aspen Acronychia acidula | 0.1-5 | 0.1-3 | 0.1-3.5 | |
| Extract Fruit | | | | |
| Manuka Honey Leptosperumum | | 0.1-6 | 0.1-2 | 0.1-4 |
| scoparium Extract Honey | | | | |
| Mountain Pepper Tasmannia | 0.1-3 | | 0.1-4 | 0.1-6 |
| lanceolata Extract Berries | | | | |
| Quandong Santalum acuminatum | 0.1-4 | 0.1-2 | | |
| Extract Fruit | | | | |
| Agrimony Agrimonia eupatoria | 0.1-5 | | | |
| Extract Herb | | | | |
| Arnica Arnica montana Extract | | | | 0.1-4 |
| Flower | | | | |
| Calendula Calendula officinalis | | 0.1-4 | | 0.1-3 |
| Extract Flower | | | | |
| Calendula Calendula officinalis | 0.1-6 | | 0.1-3 | |
| Extract Flower | | | | |
| Echinacea Echinacea purpurea | | | | 0.1-2 |
| Extract Herb | | | | |
| Green Tea Camellia sinensis | 0.1-3 | | | 0.1-4 |
| Extract Leaf | | | | |
| Rosehip Rosa canina Extract Fruit | 0.1-4 | | 0.1-2 | |
| Rosemary Rosmarinus officinalis | | | | 0.1-5 |
| Extract Leaf | | | | |
| Sage Salvia officinalis Extract | | | | 0.1-3 |
| Leaf | | | | |
| Agapanthus Agapanthus africanus | | | | 0.1-4 |
| Flower Extract Flower | | | | |
| Peach Prunus persica Extract | 0.1-6 | | 0.1-5 | |
| Fruit [symbol] | | | | |
| Water | 64-99.2 | 81-99.5 | 78.5-99.3 | 59-99 |

In some embodiments, the pH of the water used in the water-based primer of the skin scrub formulations described herein (including but not limited to Tables 1A and 1B) ranges from 4.9 to 8.1. In certain embodiments, the pH of the water used in the water-based primeris 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or 8.1.

In some embodiments, when sugar is present in a skin scrub formulation described herein, the sugar may include, but is in no way limited to, brown sugar, white sugar, granulated sugar, powdered sugar, liquid sugar, combinations thereof and the like. In addition, the sugar may be from any source, including, but in no way limited to sugar obtained from fruit (apricot, banana, peach, pear), cane, or vegetables (e.g., beet, corn, pepper, potato, corn), or animal products (e.g., lactose from milk).

In some embodiments, when coffee bean particles are present in the formulation described herein, the coffee bean may be any type of coffee bean, including, but in no way limited to Arabica or Robusta. In certain embodiments, the coffee beans used in various embodiments of the present invention have been partially or fully roasted.

In some embodiments, when one or more salt is present in the formulations described herein, including but not limited to the skin scrub formulations, the salt may include Alpenbergkern salt, Anglesey Sea salt, Alaea salt, black lava salt, brine, curing salt, Dead Sea salt, Flake salt, Fleur de sel, Halite, Himalayan salt, Kalahari salt, Kala Namak salt, Khoisan salt pearls, Kosher salt, Maras salt, Murray river salt flakes, Namibian salt pearls, Persian blue fine salt, Sal de Tavira, Sale Marino di Trapani, sea salt, Sel gris, smoked salt, Utah salt, combinations thereof and the like.

In some embodiments, the one or more oils of the skin scrub composition that include linolenic acid may include, but are in no way limited to, an oil obtained from evening primrose (Oenothera biennis), sea buckthorn seed, blackcurrant seed, borage seed, hemp seed, oats, barley, or spirulina.

In certain embodiments, the one or more oils of the skin scrub formulation that include linoleic acid may include but are in no way limited to salicornia oil, safflower oil, red raspberry seed oil, evening primrose oil, poppy seed oil, sunflower oil, barbary fig seed oil, hemp oil, corn oil, cotton seed oil, soybean oil, walnut oil, sesame oil, rice bran oil, argan oil, pistachio oil, peach pit oil, almond oil, canola oil, linseed oil, olive oil, palm oil, and coconut oil.

In various embodiments, one or more of the protecting oils described above are present in the skin scrub formulation in an amount ranging from about 1-99%, 10-90%, 20-80%, 30-70%, 40-60%, or 50-55% by weight. In some embodiments, one or more protecting oils that include ximenynic acid are present in the skin scrub formulation in an amount ranging from about 1-40%, 5-35%, 10-30%, or 15-25% by weight. In some embodiments, when oil extracted from Santalum album is one of the one or more oils in the skin scrub formulation, it is present in an amount of about 12%, 13%, 14%, 15%, 16%, 17%, 18%, 19%, or 20% by weight. In some embodiments, when an oil extracted from a Santalum obtusifolium fruit kernel is one of the one or more oils in the skin scrub formulation, it is present in an amount of about 18%, 19%, 20%, 21%, 22%, 23%, 24%, or 25% by weight. In certain embodiments, when an oil extracted from an Australian sandalwood (Santalum spicatum) nut is one of the one or more oils in the skin scrub formulation, it is present in an amount of about 5%, 6%, 7%, 8%, 9%, 10%, 11%, 12%, 13%, 14%, or 15% by weight. In some embodiments, when present in the skin scrub formulation, one or more protecting oils that include linolenic acid are present in an amount of about 1-80%, 5-75%, 10-70%, 15-65%, 20-60%, 25-55%, 30-50%, or 35-45% by weight. In some embodiments, when oil obtained from evening primrose (Oenothera biennis) is present in the skin scrub formulation, it is present in an amount of about 15%, 16%, 17%, 18%, 19%, 20%, 21%, 22%, 23%, 24%, 25%, 26%, 27%, or 28% by weight. In other embodiments, when oil obtained from evening primrose is present in the skin scrub formulation, it is present in an amount of about 50%, 51%, 52%, 53%, 54%, 55%, 56%, or 57% by weight. In some embodiments, when oil obtained from sea buckthorn seed is present in the skin scrub formulation, it is present in an amount of about 40%, 41%, 42%, 43%, 44%, 45%, 46%, 47%, 48%, 49%, 50%, 51%, 52%, 53%, 54%, 55%, 56%, 57%, 58%, 59%, or 60% by weight. In other embodiments, when oil obtained from sea buckthorn seed is present in the skin scrub formulation, it is present in an amount of about 1%, 2%, 3%, 4%, 5%, 6%, 7%, 8%, 9%, 10%, 11%, 12%, 13%, 14%, or 15% by weight. In some embodiments, when oil obtained from blackcurrant borage seed is present in the skin scrub formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight. In certain embodiments, when oil obtained from hemp seed is present in the skin scrub formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight. In some embodiments, when present in the skin scrub formulation, one or more protecting oils that include linoleic acid are present in an amount of about 1-50%, 10-40%, or 20-30% by weight. In some embodiments, when present in the skin scrub formulation, salicornia oil is present in an amount of about 30%, 31%, 32%, 33%, 34%, 35%, 36%, 37%, 38%, 39%, 40%, 41%, 42%, 43%, 44%, 45%, 46%, or 47% by weight. In certain embodiments, when present in the skin scrub formulation, safflower oil is present in an amount of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% by weight. In certain embodiments, when present in the skin scrub formulation, red raspberry seed oil is present in an amount of about 20%, 21%, 22%, 23%, 24%, 25%, 26%, 27%, 28%, 29%, 30%, 31%, 32%, 33%, 34%, 35%, 36%, 37%, 38%, 39%, or 40% by weight. In other embodiments, when present in the skin scrub formulation, red raspberry seed oil is present in an amount of .5%, 1%, 1.5%, 2%, 2.5%, 3%, 3.5%, 4%, 4.5%, 5%, 5.5%, 6%, 6.5%, 7%, 7.5%, or 8% by weight. In some embodiments, when present in the skin scrub formulation, evening primrose oil is present in an amount of about 5%, 5.5%, 6%, 6.5%, 7%, 7.5%, 8%, or 8.5% by weight. In certain embodiments, when present in the skin scrub formulation, poppy seed oil is present in an amount of about 8%, 9%, 10%, 11%, 12%, 13%, 14%, 15%, 16%, or 17% by weight. In some embodiments, when present in the skin scrub formulation, sunflower oil is present in an amount of about 40%, 41%, 42%, 43%, 44%, 45%, 46%, 47%, 48%, or 49% by weight. In some embodiments, when present in the skin scrub formulation, coconut oil is present in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight. In some embodiments, when present in the skin scrub formulation, argan oil is present in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight.

In some embodiments, in addition to the one or more protecting oils, the skin scrub formulation also includes one or more base oils. In some embodiments, one or more of the base oils are present in the formulation in an amount of about 10-60%, 20-50%, or 30-40% by weight. In some embodiments, the base oil may include, but is in no way limited to, jojoba oil, safflower oil, sunflower oil, olive oil, or combinations thereof. In certain embodiments, when present in the skin scrub formulation, jojoba oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin scrub formulation, safflower oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin scrub formulation, sunflower oil is present in an amount of about 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the skin scrub formulation, olive oil is present in an amount of about 21%, 22%, 23%, 24%, 25%, 26%, 27%, 28%, 29%, 30%, 31%, 32%, 33%, 34%, 35%, 36%, 37%, 38%, 39%, or 40% by weight.

In certain embodiments, the skin scrub formulation includes one or more protecting oils and one or more base oils and/or one or more “nutrient oils.” In some embodiments, one or more of the nutrient oils may be obtained from pomegranate seed, pumpkin seed, hazelnut, maracuja seed, blueberry seed, evening primrose, almond, rosehip, or apricot kernel. In certain embodiments, one or more of the nutrient oils are present in the skin scrub formulation in an amount of about 0.5%-10%, 3.5-7%, or 5-6% by weight. In some embodiments, when present in the skin scrub formulation, pomegranate seed oil is present in an amount of about 0.5%, 1%, 1.5%, 2%, 2.5%, 3%, 3.5%, 4%, 4.5%, 5%, 5.5%, 6%, 6.5%, 7%, 7.5%, or 8% by weight. In some embodiments, when present in the skin scrub formulation, pumpkin seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin scrub formulation, hazelnut oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, or 8.5% by weight. In some embodiments, when present in the skin scrub formulation, pumpkin seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin scrub formulation, maracuja seed oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin scrub formulation, blueberry seed oil is present in an amount of about 0.5%, 1%, 1.5%, 2%, 2.5%, 3%, 3.5%, 4%, 4.5%, 5%, 5.5%, 6%, 6.5%, 7%, or 7.5% by weight. In some embodiments, when present in the skin scrub formulation, evening primrose oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin scrub formulation, almond oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In some embodiments, when present in the skin scrub formulation, rosehip oil is present in an amount of about 0.5%, 1%, 1.5%, 2%, 2.5%, 3%, 3.5%, 4%, 4.5%, 5%, 5.5%, or 6% by weight. In some embodiments, when present in the skin scrub formulation, apricot kernel oil is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, or 9% by weight.

In certain embodiments, the skin scrub formulation includes one or more protecting oils and one or more base oils and/or one or more nutrient oils and/or one or more “essential oils.” In certain embodiments, one or more of the essential oils may contain one or more of limonene, linalool, geraniol, and citral. In some embodiments, the one or more oils that include limonene are obtained from peels of citrus plants (e.g., grapefruit, orange, lemon, tangerine, lime, pomelo, bergamot and the like). In some embodiments, the one or more oils that include linalool may include but are in no way limited to an oil obtained from coriander (Coriandrum sativum L. family Apiaceae) seed, palmarosa [Cymbopogon martinii var martinii (Roxb.) Wats., family Poaceae], sweet orange (Citrus sinensis Osbeck, family Rutaceae) flowers, lavender (Lavandula officinahs Chaix, family Lamiaceae), bay laurel (Laurus nobilis, family Lauraceae), sweet basil (Ocimum basilicum, family Lamiaceae), and bergamot. In some embodiments, the one or more oils that include geraniol may include but are in no way limited to rose oil, palmarosa oil, and citronella oil (Java type), geranium oil, and lemon oil. In some embodiments, the one or more oils that include citral may include but are in no way limited to an oil obtained from lemon myrtle, Litsea citrata, Litsea cubeba, lemongrass, lemon tea-tree, Ocimum gratissimum, Indera citriodora, Calypranthes parriculata, petitgrain, lemon verbena, ironbark, lemon balm, lime peel, lemon peel, and orange peel.

In some embodiments, one or more of the essential oils may include, but are in no way limited to, bergamot essential oil, grapefruit essential oil, citronella essential oil, and lemon myrtle essential oil. In certain embodiments, one or more of the essential oils are present in the skin scrub formulation in an amount of about 0.5%-11%, 3.5-8%, or 5-6% by weight. In some embodiments, when bergamot essential oil is present in the skin scrub formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight. In certain embodiments, when grapefruit essential oil is present in the skin scrub formulation, it is present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%. 10%, 10.5% or 11% by weight.

Certain non-limiting examples of skin scrub compositions/formulations (AV11-AV16) of various embodiments of the present disclosure are set forth below in Table 4 (all values are % weight).

TABLE 4

| Ingredient | AV11 | AV12 | AV13 | AV14 | AV15 | AV16 |
|---|---|---|---|---|---|---|
| Sea Buckthorn | 1.4 | | 3.5 | 4.6 | 2.2 | |
| seed oil | | | | | | |
| Red Raspberry | 0.6 | | | | 3.5 | 5.3 |
| seed oil | | | | | | |
| Sandalwood | 0.5 | | 4.2 | 1.8 | 1.3 | 2.7 |
| nut oil | | | | | | |
| Pomegranate | 2.15 | 3.6 | | 0.8 | | |
| seed oil | | | | | | |
| Borage seed | | 8 | | | 0.6 | |
| oil | | | | | | |
| Pumpkin seed | | | | | | |
| oil | | | | | | |
| Hazelnut oil | | | | 3.3 | 3.9 | |
| Maracuja seed | | | | | | 4.1 |
| oil | | | | | | |
| Rosehip oil | | 4 | | | | |
| Coconut oil | 9 | | | 4.5 | | |
| Coconut pulp | 1.4 | | | | | |
| extract | | | | | | |
| Blueberry seed | | | | | | |
| oil | | | | | | |
| Citronella | 0.45 | | | | | |
| essential | | | | | | |
| oil | | | | | | |
| Apricot kernel | | | | | | 8 |
| oil | | | | | | |
| Argan oil | | | | 12 | | |
| Avocado oil | | 10.5 | 18 | | 14 | |
| Jojoba oil | 9 | 26.3 | 27.3 | | | |
| Safflower oil | 23 | | | | | 10.5 |
| Bergamot | | | | | | |
| essential | | | | | | |
| oil | | | | | | |
| Grapefruit | | | | | | |
| essential | | | | | | |
| oil | | | | | | |
| Frankicense | | | | | | |
| essential oil | | | | | | |
| Salt | 9.2 | 12 | | | 17 | |
| Dry coffee | 18.3 | | 5 | 13 | | |
| bean (e.g., | | | | | | |
| ground, | | | | | | |
| crushed or | | | | | | |
| otherwise | | | | | | |
| broken | | | | | | |
| down) | | | | | | |
| Raspberry | | | | | | 28 |
| seed flour | | | | | | |
| Chapati flour | | 35.6 | 42 | 28 | 37 | |
| Brown sugar | 25.5 | | | | | |
| Rye flour | | | | 32 | 20.5 | 41.4 |
| Almond flour | | | | | | |
| TOTAL % | 100 | 100 | 100 | 100 | 100 | 100 |

In some embodiments, one or more oils of the skin scrub formulations described herein are obtained by cold pressing the nut, seed, or any other portion of a plant described herein and decanting, as described in Hanna, M. A. et al, ‘Expression of oil from oilseeds—A review’ J Agric. Eng. Res., 1998, 28 (6) 495-583, which is incorporated herein by reference in its entirety. In some embodiments, oils are extracted from the nut, seed, or any other portion of a plant described herein through the use of supercritical CO_{2,} as described in Ibanez, E. et al, ‘Sub—and supercritical fluid extraction of functional ingredients from different natural sources: Plants, food-by-products, algae and microalgae: A review’, 2006, 98(1), 136-148 which is incorporated herein by reference in its entirety. Skin scrub formulations described herein which include coffee and salt (as described above) have also been shown to improve symptoms of psoriasis and eczema in human subjects, as described in greater detail in the examples set forth herein. While all tested formulations which included coffee and salt resulted in a significant improvement in psoriasis and eczema, those which further included powdered sugar were surprisingly especially effective, resulting in even greater improvement than those tested formulations without powdered sugar.

The skin scrub and water-based primer compositions according to various embodiments described herein may be delivered topically in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic composition and the physiological condition of the subject (including, but not limited to, age, sex, disease type and stage, general physical condition, responsiveness to a given dosage, and type of composition). Merely by way of example, a subject with exceptionally dry skin my benefit from more frequent applications (e.g., 2× per week) of a greater quantity of the compositions than a subject with mildly dry skin.

### Skin Scrub and Water-based Primer Methods of Treatment

In various embodiments, the present invention provides a method for treating a subject's skin with a skin scrub formulation or water-based primer described herein. In certain embodiments, the method includes applying a skin scrub formulation or water-based primer described herein to a subject's foot. In certain embodiments, the method includes applying a skin scrub formulation or water-based primer described herein to the bottom of a subject's foot. In certain embodiments, the method includes applying a skin scrub formulation or water-based primer described herein to rough skin which may or may not include a callus. In some embodiments, the method includes applying a skin scrub formulation or water-based primer described herein to a subject's face. In certain embodiments, the method includes applying a skin scrub formulation or water-based primer described herein to a subject's face, and then applying makeup to the subject's face. In certain embodiments, a skin scrub formulation or water-based primer described herein is applied to a subject's skin before or after shaving hair on the skin.

In some embodiments, the invention teaches a method of treating a subject's skin that includes applying a skin scrub composition described herein to the subject's skin. In some embodiments, the skin scrub composition applied includes coffee, salt, and an oil described herein. In some embodiments, the skin scrub composition applied includes, coffee, salt, and safflower oil. In some embodiments, the skin scrub composition applied includes coffee, salt, and sugar. In some embodiments, the skin scrub composition applied includes coffee, salt, sugar, and an oil.

In some embodiments, the method includes applying formulation AV11 of Table 4 onto a subject's skin. In some embodiments, the method includes applying formulation AV12 of Table 4 onto a subject's skin. In some embodiments, the method includes applying formulation AV13 of Table 4 onto a subject's skin. In some embodiments, the method includes applying formulation AV14 of Table 4 onto a subject's skin. In some embodiments, the method includes applying formulation AV15 of Table 4 onto a subject's skin. In some embodiments, the method includes applying formulation AV16 of Table 4 onto a subject's skin. In certain embodiments, the subject has one or more skin condition described herein. In some embodiments, the skin condition may include one or more of dry skin, eczema, acne, dermatitis, or psoriasis.

In certain embodiments, the invention teaches a method that includes applying a water-based primer as described herein to a subject's skin and then applying a composition that includes a particulate scrubbing ingredient described or referenced herein, or combination of two or more particulate scrubbing ingredients described or referenced herein to a subject's skin. In some embodiments, the method further includes a subsequent step of rinsing off the particulate scrubbing ingredient(s) that was/were applied to the skin with water or one or more water-based primer formulations described herein. In some embodiments the composition that includes one or more scrubbing ingredients includes no additional ingredients. In other words, merely by way of example, ground coffee and/or ground raspberry seed flour and/or ground oat flour is added to directly to skin that has been previously treated with a water-based primer described herein, and none of the additional oils or other ingredients described herein are included along with the scrubbing ingredients.

In some embodiments, topical application of the skin scrub or water-based primer formulations described herein is accomplished by hand. In certain embodiments, topical application of the skin scrub or water-based primer formulations described herein is accomplished with one or more of a dropper, brush, sponge, wipe, syringe, straw, hose, and the like.

In certain embodiments, any of the skin scrub formulations described herein may be incorporated into an absorbent material, including but in no way limited to, a sponge, a wipe, a tissue, a pad, a paper, a cloth, a towel, a cotton ball, a glove, a gauze, a band-aid, a patch.

In some embodiments, a skin scrub composition as disclosed herein may be administered at the prevention stage (i.e., when the subject has not developed any of the conditions described herein, but is likely to or in the process of developing a condition described herein (e.g. acne, wrinkles, dry skin, psoriasis, dermatitis, cellulitis, eczema, etc.). In other embodiments, a skin scrub composition as disclosed herein may be administered at the treatment stage (e.g., when the subject has already developed acne, wrinkles, dry skin, psoriasis, dermatitis, cellulitis, eczema, etc.). In other embodiments, a skin scrub composition as disclosed herein may be administered at the maintenance stage (e.g., when the subject has been successfully treated for acne, wrinkles, dry skin, psoriasis, dermatitis, cellulitis, eczema, etc.).

In various embodiments, any of the skin scrub or water-based formulations described herein (e.g., those that contain one or more oils as well as those that do not) may be applied to a subject's skin 1-20 or more times per day. In various embodiments, any of the formulations may be applied to a subject's skin 1, 2, 3, 4, 5, 6, 7, 8, 9, 10 or more times per day.

### Multiphase Compositions

In various embodiments, the invention teaches a skin care protecting composition (also described herein as a “formulation” or “skin formulation”) that includes one or more “protecting oils” that include ximenynic acid. In some embodiments, the skin protecting composition may further include, but is in no way limited to, one or more additional oils chosen from borage oil, cranberry seed oil, pomegranate seed oil, palm kernel oil, hazelnut oil, sunflower seed oil, pistachio oil, peanut oil, brazil nut oil, olive oil, walnut oil, raspberry seed oil, lemon oil, orange oil, cotton seed oil, soybean oil, jojoba oil, coconut oil, sesame oil, Sea Buckthorn oil, pumpkin seed oil, and passion fruit seed oil.

In some embodiments, the one or more oils of the skin care formulation that include ximenynic acid may include, but are in no way limited to, an oil obtained from Ximenia or from Santalum sp. In some embodiments, the oil is obtained from a plant in the Santalaceae family. In some embodiments, the oil may be obtained from Ximenia Americana (also known as Mountain Plum or Manzanella or Cagalera or manzana del diablo or Tallow wood) or Ximenia Africana or Santalum spicatum or Santalum acuminatum (also known as Desert quandong or Sandalwood or Katunga or Burn-burn or Mangatais) or Santalum album. In certain embodiments one or more of the oils containing ximenynic acid are obtained from the fruit kernel of Blunt Sandalwood (Santalum obtusifolium) or the nut of an Australian Sandalwood (Santalum spicatum). In certain embodiments, one or more oils containing ximenynic acid are obtained from seed oil of other plants in the Santalaceae family including, but in no way limited to, the Native Cherry (Exocarpos cupressiformis). In some embodiments, the skin protecting composition that includes ximenynic acid is also referred to as a “phase II skin protector” or “skin protector.”

In certain embodiments, a formulation termed a “phase I skin primer” or a “skin primer” is provided. In some embodiments, the phase I skin primer includes sea buckthorn seed oil. In certain embodiments, the phase I skin primer may include, but is in no way limited to one or more additional oils chosen from cranberry seed oil, pomegranate seed oil, palm kernel oil, hazelnut oil, sunflower seed oil, pistachio oil, peanut oil, brazil nut oil, olive oil, walnut oil, raspberry seed oil, lemon oil, orange oil, cotton seed oil, soybean oil, jojoba oil, coconut oil, sesame oil, Sea Buckthorn oil, pumpkin seed oil, and passion fruit seed oil.

Table 5 below demonstrates exemplary non-limiting examplary concentrations of ingredients for formulations of phase I skin primers (SP1-SP4 represent the names of specific combinations of ingredients) in accordance with various embodiments of the invention. All values listed are % weight.

TABLE 5

| Ingredient | SP1 | SP2 | SP3 | SP4 |
|---|---|---|---|---|
| Cranberry Seed Oil | 27% | | 25% | |
| Lemon Oil | | 23% | | 21% |
| Pomegranate Seed Oil | 25% | 21% | | |
| Raspberry Seed Oil | 24% | | 28% | 30% |
| Orange Oil | | 26% | | |
| Sea Buckthorn Oil | 14% | 12% | | 18% |
| Palm Kernel Oil | | 7% | 23% | 17% |
| Hazelnut Oil | 4% | | | |
| Sunflower Seed Oil | | 3% | 6% | 2% |
| Pumpkin Seed Oil | 3% | | 7% | 7% |
| Passion fruit seed oil | 3% | | 11% | 5% |
| Peanut Oil | | 3% | | |
| Olive Oil | | 5% | | |
| Total Percent | 100% | 100% | 100% | 100% |

In some embodiments, one or more oils of the phase I skin primer described herein include linolenic acid. In some embodiments, one or more of the oils that include linolenic acid may include, but are in no way limited to, an oil obtained from evening primrose (Oenothera biennis), Sea Buckthorn seed, blackcurrant seed, hemp seed, oats, barley, spirulina, cranberry seed, raspberry seed, hazelnut, pumpkin seed, passion fruit seed, lemon seed, orange seed.

In certain embodiments, one or more oils of the phase I skin primer described herein include linoleic acid. In some embodiments, one or more of the oils that include linoleic acid may include, but are in no way limited to, salicornia oil, safflower oil, red raspberry seed oil, evening primrose oil, poppy seed oil, sunflower oil, barbary fig seed oil, hemp oil, corn oil, cotton seed oil, soybean oil, walnut oil, sesame oil, rice bran oil, argan oil, pistachio oil, peach pit oil, almond oil, canola oil, linseed oil, olive oil, palm oil, and coconut oil.

In various embodiments, one or more of the phase I skin primers described above are mixed with a phase II skin protector to form a single comprehensive skin care formulation. In some such embodiments, the phase I skin primer is present in the single comprehensive skin care formulation in an amount ranging from about 1-99%, 10-90%, 20-80%, 30-70%, 40-60%, or 50-55% by weight. In some embodiments, one or more protecting oils that include ximenynic acid are present in the phase II skin protector or single comprehensive skin care formulation in an amount ranging from about 1-40%, 5-35%, 10-30%, or 15-25% by weight. In some embodiments, when oil extracted from Santalum album is one of the one or more oils in the phase II skin protector or single comprehensive skin care formulation, it is present in an amount of about 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% by weight. In some embodiments, when an oil extracted from a Santalum obtusifolium fruit kernel is one of the one or more oils in the phase II skin protector or single comprehensive skin care formulation, it is present in an amount of about 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% by weight. In certain embodiments, when an oil extracted from an Australian sandalwood (Santalum spicatum) nut is one of the one or more oils in the phase II skin protector or single comprehensive skin care formulation, it is present in an amount of about 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% by weight. In some embodiments, when present, one or more oils that include linolenic acid are included in the phase I skin primer or single comprehensive skin care formulation in an amount of about 1-80%, 5-75%, 10-70%, 15-65%, 20-60%, 25-55%, 30-50%, or 35-45% by weight. In some embodiments, when oil obtained from evening primrose (Oenothera biennis) is present, it is included in the phase I skin primer or single comprehensive skin care formulation in an amount of about 15%, 16%, 17%, 18%, 19%, 20%, 21%, 22%, 23%, 24%, 25%, 26%, 27%, or 28% by weight. In other embodiments, when oil obtained from evening primrose is present, it is included in the phase I skin primer or single comprehensive skin care formulation in an amount of about 50%, 51%, 52%, 53%, 54%, 55%, 56%, or 57% by weight. In some embodiments, when oil obtained from Sea Buckthorn seed is present, it is included in the phase I skin primer or single comprehensive skin care formulation in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight. In some embodiments, when oil obtained from blackcurrant borage seed is present, it is included in the phase I skin primer or single comprehensive skin care formulation in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, or 48% by weight. In certain embodiments, when oil obtained from hemp seed is present, it is included in the phase I skin primer or single comprehensive skin care formulation in an amount of about.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight. In some embodiments, when present in the phase I skin primer or single comprehensive skin care formulation, one or more oils that include linoleic acid are present in an amount of about 1-50%, 10-40%, or 20-30% by weight. In some embodiments, when present, one or more oils that include linoleic acid are included in the phase I skin primer or single comprehensive skin care formulation in an amount of about 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight. In certain embodiments, when present, red raspberry seed oil is included in the phase I skin primer or single comprehensive skin care formulation in an amount of about 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight. In certain embodiments, when present, pomegranate seed oil is included in the phase I skin primer or single comprehensive skin care formulation in an amount of about 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight. In certain embodiments, when present, cranberry seed oil is included in the phase I skin primer or single comprehensive skin care formulation in an amount of about 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight. In certain embodiments, when present, hazelnut oil is included in the phase I skin primer or single comprehensive skin care formulation in an amount of about.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, or 13% by weight. In certain embodiments, when present, pumpkin seed oil is included in the phase I skin primer or single comprehensive skin care formulation in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, or 13% by weight. In certain embodiments, when present, passionfruit seed oil is included in the phase I skin primer or single comprehensive skin care formulation in an amount of about.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, or 13% by weight. In certain embodiments, when present, poppy seed oil is included in the phase I skin primer or single comprehensive skin care formulation in an amount of about 8%, 9%, 10%, 11%, 12%, 13%, 14%, 15%, 16%, or 17% by weight. In some embodiments, when present, sunflower oil is included in the phase I skin primer or single comprehensive skin care formulation in an amount of about 40%, 41%, 42%, 43%, 44%, 45%, 46%, 47%, 48%, or 49% by weight. In some embodiments, when present, coconut oil is included in the phase I skin primer or single comprehensive skin care formulation in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight. In some embodiments, when present, argan oil is included in the phase I skin primer or single comprehensive skin care formulation in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight.

In some embodiments, the phase II skin protector or comprehensive skin care formulation includes one or more base oils. In some embodiments, one or more of the base oils are present in the phase II skin protector or comprehensive skin care formulation in an amount of about 10-60%, 20-50%, or 30-40% by weight. In some embodiments, the base oil may include, but is in no way limited to, jojoba oil, safflower oil, sunflower oil, olive oil, or combinations thereof. In certain embodiments, when present in the phase II skin protector or comprehensive skin care formulation, jojoba oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present in the phase II skin protector or comprehensive skin care formulation, safflower oil is present in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present the phase II skin protector or comprehensive skin care formulation, sunflower oil is present in an amount of about 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% by weight. In some embodiments, when present the phase II skin protector or comprehensive skin care formulation, olive oil is present in an amount of about 21%, 22%, 23%, 24%, 25%, 26%, 27%, 28%, 29%, 30%, 31%, 32%, 33%, 34%, 35%, 36%, 37%, 38%, 39%, or 40% by weight.

In some embodiments, the invention teaches a phase I skin primer composition that can be applied to the skin of a mammal, including a human mammal, which includes 10-20% by weight sea buckthorn seed oil and 15-40% by weight red raspberry seed oil. In some embodiments, the composition further includes 15-35% by weight pomegranate seed oil. In some embodiments, the composition still further includes 15-35% by weight cranberry seed oil.

In certain embodiments, when the phase I skin primer composition includes sea buckthorn seed oil and red raspberry seed oil, the composition includes 10%, 11%, 12%, 13%, 14%, 15%, 16%, 17%, 18%, 19%, or 20% by weight sea buckthorn seed oil and 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight red raspberry seed oil. In some embodiments, the composition further includes 15%, 16%, 17%, 18%, 19%, 20%, 21%, 22%, 23%, 24%, 25%, 26%, 27%, 28%, 29%, 30%, 31%, 32%, 33%, 34%, or 35% by weight pomegranate seed oil. In some embodiments, the composition still further includes 15%, 16%, 17%, 18%, 19%, 20%, 21%, 22%, 23%, 24%, 25%, 26%, 27%, 28%, 29%, 30%, 31%, 32%, 33%, 34%, or 35% by weight cranberry seed oil.

In certain embodiments, the invention teaches a phase II skin protector composition that can be applied to the skin of a mammal, including a human mammal, which includes 50-75% by weight sandalwood nut oil and 20-50% by weight borage seed oil.

In some embodiments, when the phase II skin protector composition includes sandalwood nut oil and borage seed oil, the composition includes 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% by weight sandalwood nut oil and 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight borage seed oil.

In some embodiments, one or more oils of the multiphase skin care formulations described herein are obtained by cold pressing the nut, seed, or any other portion of a plant described herein and decanting, as described in Hanna, M. A. et al, ‘Expression of oil from oilseeds-A review’ J Agric. Eng. Res., 1998, 28 (6) 495-583, which is incorporated herein by reference in its entirety. In some embodiments, oils are extracted from the nut, seed, or any other portion of a plant described herein through the use of supercritical CO₂, as described in Ibanez, E. et al, ‘Sub—and supercritical fluid extraction of functional ingredients from different natural sources: Plants, food-by-products, algae and microalgae: A review’, 2006, 98(1), 136-148 which is incorporated herein by reference in its entirety.

Certain formulations described herein which contain ximenynic acid and linolenic acid and/or linoleic acid, have surprisingly and unexpectedly strong antioxidant, anti-insect, and ultraviolet radiation protection properties, while also absorbing rapidly into the skin and leaving little to no perceptible oily, greasy, or sticky residue. Daily application of these formulations resulted in a reduced appearance of fine lines and wrinkles on the faces and hands of study subjects, and has been shown to provide strong antioxidant protection along with protection against ultraviolet light. The formulations described herein have also been shown to improve symptoms of psoriasis, dermatitis, and eczema in human subjects. The aforementioned benefits are especially significant when a phase I skin primer formulation described herein that includes linolenic acid and/or linoleic acid is applied to the skin in a first step, and then a phase II formulation that includes ximenynic acid (whether included as purified ximenynic acid or in one or more oils that naturally contain ximenynic acid as described above) and/or borage oil is subsequently applied. In certain embodiments, the phase I skin primer formulation and the phase II skin protector formulation are applied simultaneously, or the phase II skin protector formulation may be applied prior to the phase I skin primer formulation.

The skin care compositions according to various embodiments described herein may be delivered topically in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic composition and the physiological condition of the subject (including, but not limited to, age, sex, disease type and stage, general physical condition, responsiveness to a given dosage, and type of composition).

### Multiphase Skincare Methods of Treatment

In various embodiments, the present invention provides a method for treating a subject's skin or hair by applying a phase I skin primer formulation described herein to the subject's skin or hair. In some embodiments, the invention provides a method for treating a subject's skin or hair by applying a phase II skin protector formulation described herein to the subject's skin or hair. In certain embodiments, the present invention provides a method for treating a subject's skin or hair by first applying a phase I skin primer formulation described herein to the subject's skin or hair, and then subsequently applying a phase II skin protector formulation described herein to the subject's skin or hair. In certain embodiments, the present invention provides a method for treating a subject's skin or hair by first applying phase II skin protector formulation described herein to the subject's skin or hair, and then subsequently applying a phase I skin primer formulation described herein to the subject's skin or hair. In various embodiments, the present invention provides a method for treating a subject's skin or hair by applying a phase I skin primer formulation described herein and a phase II skin protector described herein to a subject's skin or hair in any order, or simultaneously.

In certain embodiments, the invention teaches a method of treating a subject's skin that includes applying a phase I skin primer formulation that includes sea buckthorn seed oil and red raspberry seed oil to the subject's skin. In some embodiments, the phase I skin primer further includes pomegranate seed oil. In some embodiments, the method further includes applying a phase II skin protector to the subject's skin. In some embodiments, the phase II skin protector includes sandalwood nut oil and borage seed oil.

In some embodiments, topical application of the formulations described herein is accomplished by hand. In certain embodiments, topical application of the formulations described herein is accomplished with one or more of a dropper, brush, sponge, wipe, syringe, straw, hose, and the like.

In certain embodiments, any of the formulations described herein may be incorporated into an absorbent material, including but in no way limited to, a sponge, a wipe, a tissue, a pad, a paper, a cloth, a towel, a cotton ball, a glove, a gauze, a Band-Aid®, a patch.

In some embodiments, a phase I or phase II composition as disclosed herein may be administered at the prevention stage (i.e., when the subject has not developed any of the conditions described herein, but is likely to or in the process of developing a condition described herein (e.g. acne, wrinkles, dry skin, psoriasis, dermatitis, cellulitis, eczema, etc.). In other embodiments, a phase I or phase II composition as disclosed herein may be administered at the treatment stage (e.g., when the subject has already developed acne, wrinkles, dry skin, psoriasis, dermatitis, cellulitis, eczema, etc.). In other embodiments, a phase I or phase II composition as disclosed herein may be administered at the maintenance stage (e.g., when the subject has been successfully treated for acne, wrinkles, dry skin, psoriasis, dermatitis, cellulitis, eczema, etc.).

Ingredients of the same or two or more compositions of the present invention can be applied in separate steps or simultaneously depending on the type of container used. Merely by way of example, a formulation that includes sea buckthorn seed oil (e.g. in a concentration within a range described above, or as the only ingredient) and/or raspberry seed oil (e.g. in a concentration within a range described above, or as the only ingredient) may be applied in a first step, and then a formulation that includes sandalwood nut oil (e.g. in a concentration within a range described above, or as the only ingredient) may be applied in a subsequent step (e.g. in a second step). The two or more formulations can be dispensed from physically separate packages or from a unitary package with chambers. Examples of packages include, but are not limited to a pouch inside of a pouch, or a dual bladder system inside of a can. The components of either type of packages can be applied simultaneously or substantially simultaneously to the skin, where they commingle. In some embodiments, in the first step, one formulation is applied to the skin and in the second step, the other formulation is applied over the first component within a period of 1 second or less to 1 hour or more. The components are, thus, applied substantially simultaneously such that commingling occurs when the second component is applied on top of the first component.

### Kits of the Invention

In various embodiments, the present invention provides a kit for treating one or more of the conditions described herein in a subject. The kit consists of or consists essentially of or comprises: one or more compositions as described herein; and instructions for using the one or more compositions to treat the one or more conditions in the subject. In various embodiments, one, two, three, or more compositions described herein are provided.

In various embodiments, the kit is an assemblage of materials or components, including at least one of the inventive compositions described herein. In one embodiment, the kit consists of or consists essentially of or comprises a composition described herein.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, containers, spray bottles or cans, applicators (for example, applicators of scrub, cream, lotion, oil, etc., such as any of the applicators described or referenced herein, including those into which one or more of the formulations have been absorbed (e.g., wipes, sponges, etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the compositions can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in skin care therapies. As used herein, the term “package” refers to a suitable solid matrix or material such as glass, plastic, paper, foil, and the like, capable of holding the individual kit components. Thus, for example, a package can be a glass or plastic vial used to contain suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive methods, compositions, kits, and systems, and the various conditions, diseases, and disorders that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The following examples are offered for illustrative purposes only, and are not intended to limit the scope of the present invention in any way.

### Example 1

### Acne Treatment

A skin care formulation that included sandalwood nut oil and sea buckthorn oil was applied liberally one time per night to the faces of 3 healthy human subjects (ages 13 (female), 15 (female), and 44 (male)) with active acne lesions. After 2 weeks of use, all 3 subjects had 20-50% fewer acne lesions. In addition, in all cases, remaining acne lesions were significantly diminished in size and redness. The 44-year-old male had significantly less noticeable wrinkles after 2 weeks of treatment, and all subjects noted significantly softer feeling skin after 2 weeks of treatment.

### Example 2

### Antioxidant and Protective Attributes of Composition

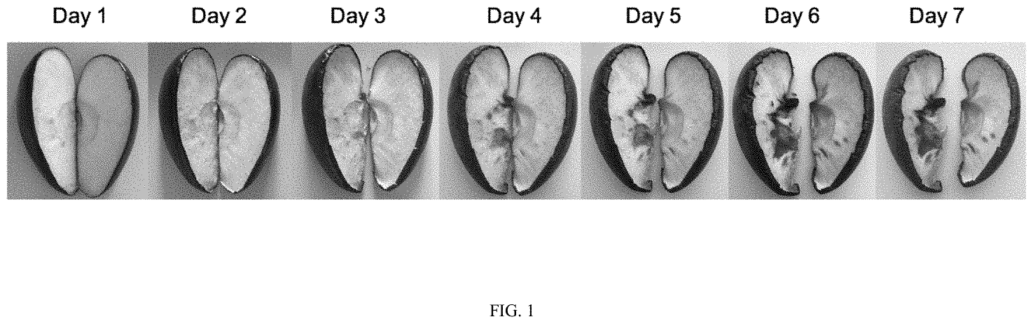

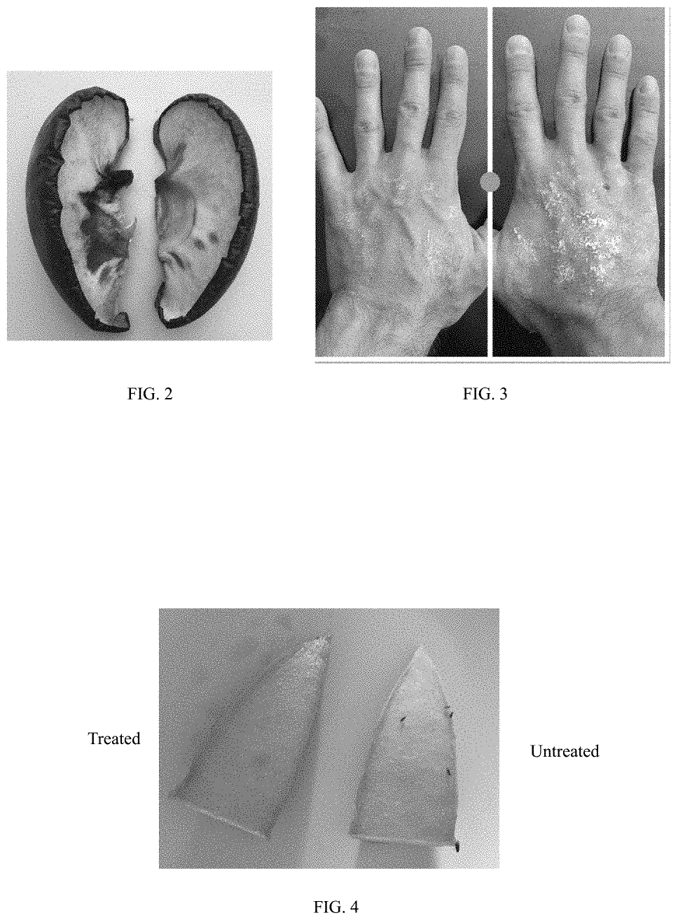

Two slices of the same apple were extracted for use. The first slice of apple was treated once with an oil-based skin care formulation containing sandalwood nut oil, sea buckthorn seed oil, and red raspberry seed oil. The second slice was not treated. FIG. 1 demonstrates a 6-day time lapse comparison between a slice of the apple that was untreated (left side of each photograph) and a slice of the apple that was treated once on day 1 with an oil-based skin care formulation containing sandalwood nut oil, sea buckthorn seed oil, and red raspberry seed oil (right side of each photograph). The progression over time is from left to right. It is evident that the untreated slice of apple oxidized and degraded through microbial action much more quickly than the slice of apple treated with the protective skin care formulation. FIG. 2 depicts a closer view of the two apple slices from day 6 of the experiment described above. The oxidation and degradation of the untreated apple slice on the left can be clearly seen. The apple slice on the right has been largely protected from oxidation and degradation.

### Example 3

FIG. 3 shows a comparison between one hand (left) that was treated with an oil-based skin care formulation containing oils that collectively contained ximenynic acid, gamma linolenic acid, and linoleic acid, and another hand (right) that was treated with the same amount of coconut oil. After 20 seconds, each hand was covered evenly with the same amount of flour. Subsequently, hands were shaken, inverted, and lightly rubbed to remove excess. It is clear that the hand on the left had significantly less flour remaining, indicating that the skin care formulation applied to the hand on the left absorbed into the hand much more quickly and left less sticky residue than the formulation applied to the right hand. The same experiment was repeated using jojoba oil, argan oil, and confectioner sugar as controls, affording similar results. Using each of these experiments, the hand with the oil-based skin care formulation containing oils that collectively contained ximenynic acid, gamma linolenic acid, and linoleic acid resulted in less flour sticking to the hand than with jojoba oil, argan oil, and confectioner sugar.

### Example 4

FIG. 4 shows a comparison between a slice of cantaloupe treated with an oil-based skin care composition containing sandalwood nut oil, sea buckthorn seed oil, and red raspberry seed oil and one untreated slice. It is evident that the treated slice displays insect repellant properties. For the treated slice, a few drops of the skin care composition was applied on the surface, then it was not touched again. During observation of the slices over the course of 5 days, a group of 5 fruit flies landed periodically on the slice that was not treated. However, flies never landed on the slice that was treated.

### Example 5

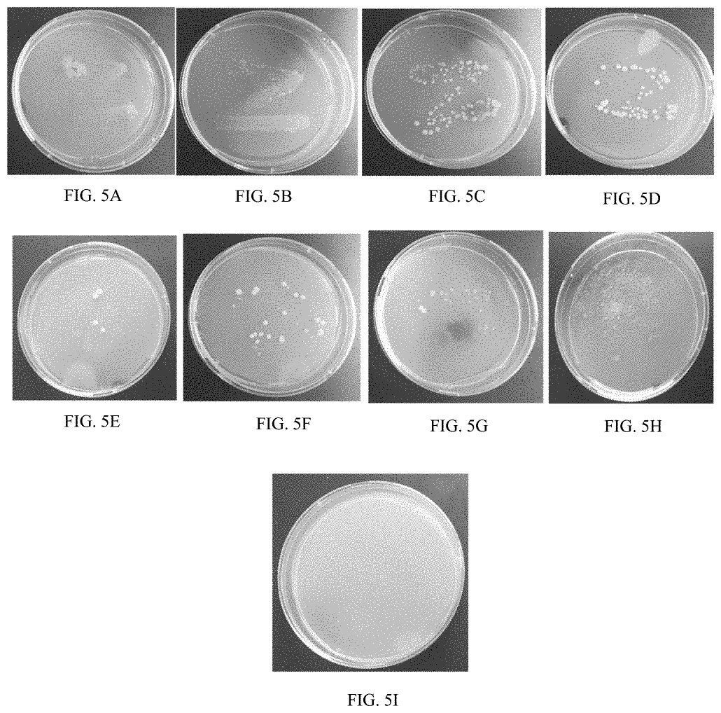

FIGS. 5A-5I show results of bacterial growth experiments on agar plates, demonstrating the antimicrobial effect of ingredients included in certain formulations described herein. In each of the agar plates shown, the following procedure was performed. An index finger was thoroughly washed with water and dried. The finger was rubbed vigorously back and forth 3 times on the surface of a dirty kitchen floor that had not been cleaned for 24 hours. For each experiment the index finger was subsequently treated with ingredients as follows: A) sea buckthorn seed oil; B) sandalwood seed oil (also known as Sandalwood nut oil); C) tea tree oil; D) frankincense oil; E) geranium oil; F) grapefruit oil; and G) lavender oil. In a control experiment, represented in H) the contaminated finger wasn't treated with any product. In the last experiment, represented in I), the finger was treated with a formulation containing all of the ingredients of A-G, in accordance with an embodiment of the invention. For each of A-I, the finger was rubbed gently (streaked) onto an agar plate producing the letter ‘Z’, and the agar plates were each quickly placed in an incubator at 38 C. Photographs were taken at 24 hour intervals. The images shown in FIGS. 5A-5I were taken 96 hours (4 days) after streaking was completed. After 96 hours, it is clear that the untreated finger showed the highest contamination (bacteria spread all over the plate). Each ingredient showed different levels of antimicrobial properties, with geranium oil being the most effective, and sandalwood seed oil being the least effective. The finger treated with the formulation that included all ingredients of A-G did not result in any growth, as can be seen in FIG. 51 Several controls experiments were performed: each ingredient was deposited onto an agar plate and placed in an incubator at 38 C for 3 days, but they didn't produce any growth. This demonstrates that the bacterial growth came from the finger and not from the ingredients.

### EXAMPLE 6

### Skin Penetration Enhancement

Five drops of a skin care formulation that included red raspberry seed oil was applied to the left side of the face of 5 healthy volunteers (M 39, M 44, F 15, F13, F 43). 5 drops of a skin care formulation that included sandalwood nut oil and borage seed oil were applied to the right side of the face of the same 5 healthy volunteers. After 30 seconds, 5 drops of the skin care formulation that were applied to the right side of the face of the 5 healthy volunteers were also applied to the left side of the face of the same 5 healthy volunteers. After 10 minutes, the left side of the faces of each of the healthy volunteers were visibly less greasy, because red raspberry seed oil facilitated significantly faster absorption and deeper penetration of the skin care formulation that included sandalwood nut oil and borage seed oil only. A tissue was subsequently applied to each side of the subject's faces, and revealed significantly less residue on the left side, which was pretreated with red raspberry seed oil. For the next 4 weeks, on a nightly basis, each of the 5 healthy subjects applied a commercially available moisturizer on the right side of their faces. Meanwhile, the formulation that included the red raspberry seed oil was applied each night to the left side of their faces. 1 minute later, each night, the formulation that includes sandalwood nut oil and borage seed oil was also applied to the left side of their faces. After 4 weeks, all subjects had visibly less pronounced facial wrinkles, and perceived softer skin on the left side of their faces. Additionally, 3 out of the 5 subjects had fewer acne skin lesions (the other two subjects had no acne lesions at the beginning of the study).

### Example 7

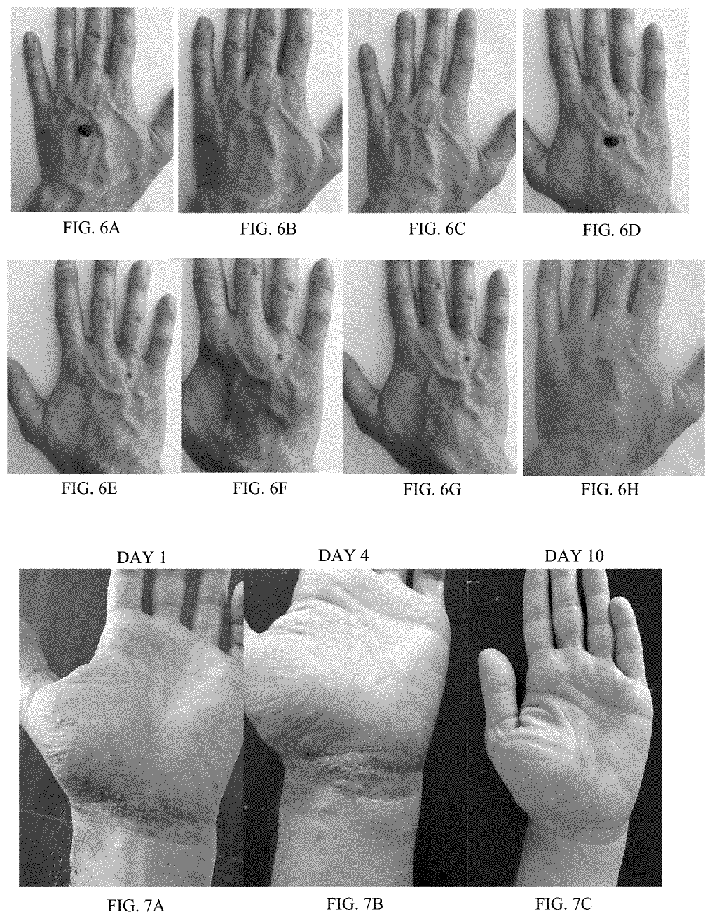

FIGS. 6A-6H show images demonstrating the degree of penetration of water-based dye onto hands. 6A) A hand treated with a composition (“combined formulation”) that includes a phase I skin primer that contains Sea Buckthorn oil and a phase II skin protectant that includes borage seed oil and sandalwood nut oil. A drop of pink food dye was then applied to the hand. 6B) The same hand as in A after wiping the dye away with a paper towel. 6C) The hand treated in 6A, then wiped with a paper towel in 6B, and subsequently washed with water and soap. 6D) A hand treated with a commercially available oil-based moisturizer, and then a drop of pink food dye was applied to the hand. 6E) the same hand in 6D after wiping the dye with a paper towel in the same manner as the hand treated in 6B. 6F) A hand first treated with a commercially available water-based moisturizer, then pink food dye was applied to the hand, and finally the hand was wiped with a paper towel. 6G) A hand treated with a commercially available water-based moisturizer, then pink food dye was applied, and finally the hand was washed with soap and water. 6H) A hand treated with the combined formulation described in 6A. A drop of pink food dye was applied to the hand 8 hours after the combined formulation was applied, and then the hand was wiped with a paper towel. The combined formulation significantly prevented penetration of the dye even 8 hours after the combined formulation was applied. In the experiments in FIGS. 6A-6H described above, in each case the hand was first washed with soap and water and then about 1 minute after application of each product (8 hours for the experiment shown in 6H), a drop of pink food dye was deposited in the center of the hand. Immediately after depositing the drop, the dye was spread quickly with one circular motion around the skin and then immediately removed using a paper towel by scrubbing the skin vigorously. A control experiment was performed on an untreated clean hand. These experiments demonstrated that oil-based products produced the highest level of protection by preventing the dye from penetrating the skin, because, using a minimum amount of water, it was possible to remove the dye completely. Although the effect of the combined formulation of phase I skin primer and phase II skin protectant (“combined formulation”) was similar to the commercially available oil-based product, the combined formulation showed a visibly superior hydrophobic effect, and the commercially available oil-based product was significantly greasier, whereas the combined formulation was completely invisible to the eye and to touch. A water-based commercially available product produced a worse result than an untreated hand.

### Example 8

### Contact Dermatitis Treatment

A subject (M 39) presented with contact dermatitis of unknown cause (FIG. 7A). The subject was treated once daily by applying a skin care composition that includes sandalwood nut oil, sea buckthorn, and red raspberry seed oil to the affected wrist area. Significant improvement was noticeable after 2 days of daily treatment. FIG. 7B shows the subject's wrist after 3 days of treatment. FIG. 7C shows the subject's wrist after 9 days of treatment.

### Example 9

### Eczema Treatment

2 subjects (M 43, F 33) with eczema applied a skin scrub composition including ground coffee, sea salt, and sugar to skin lesions 1 time per day in the morning. After 3 days of treatment there was a significant difference in the appearance of the eczema lesions. After 2 weeks of treatment, the eczema lesions were barely noticeable in either subject.

### Example 10

### Psoriasis Treatment

1 subject (F 32) with more than 50 significant and highly inflamed psoriasis lesions on her back applied a skin care composition including sea buckthorn seed oil and sandalwood nut oil once daily for 5 days to the lower right quadrant of her back, while applying a moisturizing cream to the lower left quadrant of her back. The subject experienced significant relief from itching on both sides of her back after 3 days of treatment, and the lesions were noticeably smaller and less red on the right side of her back after 5 days of treatment.

### Example 11

### 2,2-diphenyl-1-picryhydrazyl radical (DPPH) Scavenging Experiment

DPPH is a stable radical, which was reportedly used to estimate the radical-scavenging capacities of antioxidants and to evaluate the kinetics and thermodynamic properties of radical-antioxidant reactions (Yu et al., Journal of agricultural and food chemistry (2002) 50, 1619-1624).

In order to test ingredients in embodiments of the present invention, DPPH scavenging capacity was estimated according to the procedure previously described in Yu et al., 2002. Freshly prepared DPPH solution was mixed with each of the antioxidant oils to start the radical-antioxidant reaction. The final concentration was 127 micromolar for DPPH, and the total volume was 2.0 mL for each reaction mixture. The absorbance at 517 nm was determined against a blank of pure ethanol at 0, 1, 5, 10, 20, and 40 minutes of reaction and used to estimate the remaining radical levels according to a standard curve. The time-dependencies of the oil extracts and DPPH reactions were demonstrated by plotting the percent of DPPH remaining against time for each oil extract or combinations of oil extracts tested. Among other oils, in this study we individually tested the antioxidant properties of cold-pressed sea buckthorn seed oil, cold-pressed red raspberry seed oil, and sandalwood nut oil extracted with supercritical CO₂. We then tested the antioxidant properties of a mixture of sea buckthorn seed oil, red raspberry seed oil, and sandalwood nut oils at 55%, 33%, and 12% w/w respectively (COMBO1). All of the seed and nut oils directly reacted with and quenched DPPH radicals. Notably, COMBO1 displayed significantly stronger antioxidant activities than the individual components, thus demonstrating a surprisingly robust synergistic effect.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

l-

21

. (canceled)

22. A skin care composition comprising coffee beans and geraniol.

23. The skin care composition of claim 22 further comprising sugar.

24. The skin care composition of claim 23, wherein the sugar is powdered sugar.

25. The skin care composition of claim 24 further comprising an oil.

26. The skin care composition of claim 25, wherein the oil is safflower oil.

27. The skin care composition of claim 26 further comprising salt.

28. A method of treating dry skin of a subject in need thereof, comprising applying a skin care composition comprising coffee beans and geraniol to the subject's skin.

29. The method of claim 28, wherein the composition further comprises sugar.

30. The method of claim 29, wherein the sugar is powdered sugar.

31. The method of claim 28, wherein the dry skin is caused by psoriasis.

32. The method of claim 28, wherein the dry skin is caused by eczema.

33. A method of preventing psoriasis in a subject in need thereof, comprising applying a skin care composition comprising coffee beans and geraniol to the subject's skin.

34. The method of claim 33, wherein the composition further comprises sugar.

35. The method of claim 34, wherein the sugar is powdered sugar.
